(19) United States
(12) Patent Application Publication
(10) Pub. No.: US 2019/0365710 A1
(43) Pub. Date: Dec. 5, 2019

(54) METHODS OF TREATING CANCER

(71) Applicant: GlaxoSmithKline Intellectual Property Development Limited, Brentford, Middlesex (GB)

(72) Inventors: Andy FEDORIW, Collegeville, PA (US); Sarah GERHART, Collegeville, PA (US); Ryan G. KRUGER, Collegeville, PA (US); Jenny LARAIO, Collegeville, PA (US); Helai MOHAMMAD, Collegeville, PA (US); Shane W. OBRIEN, Collegeville, PA (US); Jacob RUBIN, Collegeville, PA (US)

(21) Appl. No.: 16/465,630

(22) PCT Filed: Nov. 30, 2017
(86) PCT No.: PCT/IB2017/057550

§ 371 Date: May 31, 2019

Related U.S. Application Data
(60) Provisional application No. 62/428,780, filed on Dec. 1, 2016.

(51) Int. Cl. A61K 31/415; A61P 35/00; C12Q 1/6886; A61K 45/06

(52) U.S. Cl. CPC A61K 31/415; A61P 35/00; C12Q 2600/158; A61K 45/06; C12Q 2600/106; C12Q 1/6886

(57) ABSTRACT

This invention relates to methods of treating cancer in a subject in need thereof, e.g., in a human in need thereof, comprising determining the level of 5-Methylthioadenosine phosphorylase (MTAP) polynucleotide or polypeptide or the presence or absence of a mutation in MTAP in a sample from the human, and administering to the human an effective amount of a Type I protein arginine methyltransferase (Type I PRMT) inhibitor if the level of the MTAP polynucleotide or polypeptide is decreased relative to a reference or if a mutation in MTAP polynucleotide or polypeptide is present, thereby treating the cancer in the human.

### FIELD OF THE INVENTION

This invention relates to methods of treating cancer in a subject in need thereof.

### BACKGROUND OF THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

The expanding development and use of targeted therapies for cancer treatment reflects an increasing understanding of key oncogenic pathways, and how the targeted perturbation of these pathways corresponds to clinical response. Difficulties in predicting efficacy to targeted therapies is likely a consequence of the limited global knowledge of causal mechanisms for pathway deregulation (e.g. activating mutations, amplifications). Pre-clinical translational research studies for oncology therapies focuses on determining what tumor type and genotypes are most likely to benefit from treatment. Treating selected patient populations may help maximize the potential of a therapy. Pre-clinical cellular response profiling of tumor models has become a cornerstone in development of novel cancer therapeutics.

Arginine methylation is an important post-translational modification on proteins involved in a diverse range of cellular processes such as gene regulation, RNA processing, DNA damage response, and signal transduction. Proteins containing methylated arginines are present in both nuclear and cytosolic fractions suggesting that the enzymes that catalyze the transfer of methyl groups on to arginines are also present throughout these subcellular compartments (reviewed in Yang, Y. & Bedford, M. T. Protein arginine methyltransferases and cancer. Nat Rev Cancer 13, 37-50, doi:10.1038/nrc3409 (2013); Lee, Y. H. & Stallcup, M. R. Minireview: protein arginine methylation of nonhistone proteins in transcriptional regulation. Mol Endocrinol 23, 425-433, doi:10.1210/me.2008-0380 (2009)). In mammalian cells, methylated arginine exists in three major forms: ω-N^{G}-monomethyl-arginine (MMA), ω-N^{G},N^{G}-asymmetric dimethyl arginine (ADMA), or ω-N^{G},N′^{G}-symmetric dimethyl arginine (SDMA). Each methylation state can affect protein-protein interactions in different ways and therefore has the potential to confer distinct functional consequences for the biological activity of the substrate (Yang, Y. & Bedford, M. T. Protein arginine methyltransferases and cancer. Nat Rev Cancer 13, 37-50, doi:10.1038/nrc3409 (2013)).

Arginine methylation occurs largely in the context of glycine-, arginine-rich (GAR) motifs through the activity of a family of Protein Arginine Methyltransferases (PRMTs) that transfer the methyl group from S-adenosyl-L-methionine (SAM) to the substrate arginine side chain producing S-adenosyl-homocysteine (SAH) and methylated arginine. This family of proteins is comprised of 10 members of which 9 have been shown to have enzymatic activity (Bedford, M. T. & Clarke, S. G. Protein arginine methylation in mammals: who, what, and why. Mol Cell 33, 1-13, doi:10.1016/j.molcel.2008.12.013 (2009)). The PRMT family is categorized into four sub-types (Type I-IV) depending on the product of the enzymatic reaction. Type IV enzymes methylate the internal guanidino nitrogen and have only been described in yeast (Fisk, J. C. & Read, L. K. Protein arginine methylation in parasitic protozoa. Eukaryot Cell 10, 1013-1022, doi:10.1128/EC.05103-11 (2011)); types I-III enzymes generate monomethyl-arginine (MMA, Rme1) through a single methylation event. The MMA intermediate is considered a relatively low abundance intermediate, however, select substrates of the primarily Type III activity of PRMT7 can remain monomethylated, while Types I and II enzymes catalyze progression from MMA to either asymmetric dimethyl-arginine (ADMA, Rme2a) or symmetric dimethyl arginine (SDMA, Rme2s) respectively. Type II PRMTs include PRMT5, and PRMT9, however, PRMT5 is the primary enzyme responsible for formation of symmetric dimethylation. Type I enzymes include PRMT1, PRMT3, PRMT4, PRMT6 and PRMT8. PRMT1, PRMT3, PRMT4, and PRMT6 are ubiquitously expressed while PRMT8 is largely restricted to the brain (reviewed in Bedford, M. T. & Clarke, S. G. Protein arginine methylation in mammals: who, what, and why. Mol Cell 33, 1-13, doi:10.1016/j.molcel.2008.12.013 (2009)).

Mis-regulation and overexpression of PRMT1 has been associated with a number of solid and hematopoietic cancers (Yang, Y. & Bedford, M. T. Protein arginine methyltransferases and cancer. Nat Rev Cancer 13, 37-50, doi:10.1038/nrc3409 (2013); Yoshimatsu, M. et al. Dysregulation of PRMT1 and PRMT6, Type I arginine methyltransferases, is involved in various types of human cancers. Int J Cancer 128, 562-573, doi:10.1002/ijc.25366 (2011)). The link between PRMT1 and cancer biology has largely been through regulation of methylation of arginine residues found on relevant substrates. In several tumor types, PRMT1 can drive expression of aberrant oncogenic programs through methylation of histone H4 (Takai, H. et al. 5-Hydroxymethylcytosine plays a critical role in glioblastomagenesis by recruiting the CHTOP-methylosome complex. Cell Rep 9, 48-60, doi:10.1016/j.celrep.2014.08.071 (2014); Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Zhao, X. et al. Methylation of RUNX1 by PRMT1 abrogates SIN3A binding and potentiates its transcriptional activity. Genes Dev 22, 640-653, doi:10.1101/gad.1632608 (2008), as well as through its activity on non-histone substrates (Wei, H., Mundade, R., Lange, K. C. & Lu, T. Protein arginine methylation of non-histone proteins and its role in diseases. Cell Cycle 13, 32-41, doi:10.4161/cc.27353 (2014)). In many of these experimental systems, disruption of the PRMT1-dependent ADMA modification of its substrates decreases the proliferative capacity of cancer cells (Yang, Y. & Bedford, M. T. Protein arginine methyltransferases and cancer. Nat Rev Cancer 13, 37-50, doi:10.1038/nrc3409 (2013)).

Type 1 PRMT inhibitors that are useful in treating cancer have been reported in PCT application PCT/US2014/029710, which is incorporated by reference herein. It is desirable to identify genotypes that are more likely to respond to these compounds.

### SUMMARY OF THE INVENTION

In one embodiment, the present invention provides methods for treating cancer in human in need thereof, comprising: determining

a. the level of 5-Methylthioadenosine phosphorylase (MTAP) polynucleotide or polypeptide or

b. the presence or absence of a mutation in MTAP in a sample from the human, and

administering to the human an effective amount of a Type I protein arginine methyltransferase (Type I PRMT) inhibitor if the level of the MTAP polynucleotide or polypeptide is decreased relative to a control or if a mutation in MTAP polynucleotide or polypeptide is present, thereby treating the cancer in the human.

In one embodiment, the present invention provides a method of inhibiting proliferation of a cancer cell in a human in need thereof, the method comprising administering to the human an effective amount of a Type I protein arginine methyltransferase (Type I PRMT) inhibitor, thereby inhibiting proliferation of the cancer cell in the human, wherein the cancer cell has a mutation in 5-Methylthioadenosine phosphorylase (MTAP) and/or a decreased level of a MTAP polynucleotide or polypeptide relative to a control.

In one embodiment, the present invention provides to a method of predicting whether a human having cancer will be sensitive to treatment with a Type I protein arginine methyltransferase (Type I PRMT) inhibitor, the method comprising determining
a. the level of 5-Methylthioadenosine phosphorylase (MTAP) polynucleotide or polypeptide or
b. the presence or absence of a mutation in MTAP in a sample from the human,
   wherein a decreased level of MTAP polynucleotide or polypeptide relative to a control or the presence of a mutation in MTAP indicates the human will be sensitive to treatment with a Type 1 PRMT inhibitor.

In one embodiment, the present invention provides a kit for the treatment of cancer, the kit comprising an agent that specifically binds a 5-Methylthioadenosine phosphorylase (MTAP) polynucleotide or polypeptide.

In one embodiment, a pharmaceutical composition is provided, comprising a Type I PRMT inhibitor or a pharmaceutically acceptable salt thereof, for use in treating cancer in a human wherein at least a first sample from the human is determined to have a mutation in MTAP, an decreased level of level of MTAP polynucleotide or polypeptide relative to a control, or both.

In one embodiment, the present invention provides use of a Type I PRMT inhibitor in the manufacture of a medicament for the treatment of cancer in a human wherein one or more samples from the human is determined to have a mutation in MTAP, a decreased level of MTAP polynucleotide or polypeptide relative to a control, or both.

### BRIEF DESCRIPTION OF THE DRAWINGS

FIG. 1: Types of methylation on arginine residues. From Yang, Y. & Bedford, M. T. Protein arginine methyltransferases and cancer. Nat Rev Cancer 13, 37-50, doi:10.1038/nrc3409 (2013).

FIG. 2: Functional classes of cancer relevant PRMT1 substrates. Known substrates of PRMT1 and their association to cancer related biology (Yang, Y. & Bedford, M. T. Protein arginine methyltransferases and cancer. Nat Rev Cancer 13, 37-50, doi:10.1038/nrc3409 (2013); Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Wei, H., Mundade, R., Lange, K. C. & Lu, T. Protein arginine methylation of non-histone proteins and its role in diseases. Cell Cycle 13, 32-41, doi:10.4161/cc.27353 (2014); Boisvert, F. M., Rhie, A., Richard, S. & Doherty, A. J. The GAR motif of 53BP1 is arginine methylated by PRMT1 and is necessary for 53BP1 DNA binding activity. Cell Cycle 4, 1834-1841, doi:10.4161/cc.4.12.2250 (2005); Boisvert, F. M., Dery, U., Masson, J. Y. & Richard, S. Arginine methylation of MRE11 by PRMT1 is required for DNA damage checkpoint control. Genes Dev 19, 671-676, doi:10.1101/gad.1279805 (2005); Zhang, L. et al. Cross-talk between PRMT1-mediated methylation and ubiquitylation on RBM15 controls RNA splicing. Elife 4, doi:10.7554/eLife.07938 (2015); Snijders, A. P. et al. Arginine methylation and citrullination of splicing factor proline- and glutamine-rich (SFPQ/PSF) regulates its association with mRNA. RNA 21, 347-359, doi:10.1261/ma.045138.114 (2015); Liao, H. W. et al. PRMT1-mediated methylation of the EGF receptor regulates signaling and cetuximab response. J Clin Invest 125, 4529-4543, doi:10.1172/JCI82826 (2015); Ng, R. K. et al. Epigenetic dysregulation of leukaemic HOX code in MLL-rearranged leukaemia mouse model. J Pathol 232, 65-74, doi:10.1002/path.4279 (2014); Bressan, G. C. et al. Arginine methylation analysis of the splicing-associated SR protein SFRS9/SRP30C. Cell Mol Biol Lett 14, 657-669, doi:10.2478/si 1658-009-0024-2 (2009)).

FIG. 3: Methylscan evaluation of cell lines treated with Compound D. Percent of proteins with methylation changes (independent of directionality of change) are categorized by functional group as indicated.

FIG. 4: Mode of inhibition against PRMT1 by Compound A. IC₅₀ values were determined following a 18 minute PRMT1 reaction and fitting the data to a 3-parameter dose-response equation. (A) Representative experiment showing Compound A IC₅₀ values plotted as a function of [SAM]/Kₘ^{app} fit to an equation for uncompetitive inhibition IC₅₀=Kᵢ/(1+(Kₘ/[S])). (B) Representative experiment showing IC₅₀ values plotted as a function of [Peptide]/Kₘ^{app}. Inset shows data fit to an equation for mixed inhibition to evaluate Compound A inhibition of PRMT1 with respect to peptide H4 1-21 substrate (v=Vₘₐₓ*[S]/(Kₘ*(1+[I]/Kᵢ)+[S]*(1+[I]/K′))). An alpha value (α=Kᵢ′/Kᵢ)>0.1 but <10 is indicative of a mixed inhibitor.

FIG. 5: Potency of Compound A against PRMT1. PRMT1 activity was monitored using a radioactive assay run under balanced conditions (substrate concentrations equal to Kₘ^{app}) measuring transfer of ³H from SAM to a H4 1-21 peptide. IC₅₀ values were determined by fitting the data to a 3-parameter dose-response equation. (A) IC₅₀ values plotted as a function of PRMT1:SAM:Compound A-tri-HCl preincubation time. Open and filled circles represent two independent experiments (0.5 nM PRMT1). Inset shows a representative IC₅₀ curve for Compound A-tri-HCl inhibition of PRMT1 activity following a 60 minute PRMT1:SAM:Compound A-tri-HCl preincubation. (B) Compound A inhibition of PRMT1 categorized by salt form. IC₅₀ values were determined following a 60 minute PRMT1:SAM:Compound A preincubation and a 20 minute reaction.

FIG. 6: The crystal structure resolved at 2.48 Å for PRMT1 in complex with Compound A (orange) and SAH (purple). The inset reveals that the compound is bound in the peptide binding pocket and makes key interactions with PRMT1 sidechains.

FIG. 7: Inhibition of PRMT1 orthologs by Compound A. PRMT1 activity was monitored using a radioactive assay run under balanced conditions (substrate concentrations equal to Kₘ^{app}) measuring transfer of ³H from SAM to a H4 1-21 peptide. IC₅₀ values were determined by fitting the data to a 3-parameter dose-response equation. (A) IC₅₀ values plotted as a function of PRMT1:SAM:Compound A preincubation time for rat (∘) and dog (●) orthologs. (B) IC₅₀ values plotted as a function of rat (∘), dog (●) or human (□) PRMT1 concentration. (C) IC₅₀ values were determined following a 60 minute PRMT1:SAM:Compound A preincubation and a 20 minute reaction. Data is an average from testing multiple salt forms of Compound A. Kᵢ*^{app} values were calculated based on the equation Kᵢ=IC₅₀/(+(Kₘ/[S])) for an uncompetitive inhibitor and the assumption that the IC₅₀ determination was representative of the ESI* conformation.

FIG. 8: Potency of Compound A against PRMT family members. PRMT activity was monitored using a radioactive assay run under balanced conditions (substrate concentrations at Kₘ^{app}) following a 60 minute PRMT:SAM:Compound A preincubation. IC₅₀ values for Compound A were determined by fitting data to a 3-parameter dose-response equation. (A) Data is an average from testing multiple salt forms of Compound A. Kᵢ*^{app} value were calculated based on the equation Kᵢ=IC₅₀/(1+(Kₘ/[S])) for an uncompetitive inhibitor and the assumption that the IC₅₀ determination was representative of the ESI* conformation. (B) IC₅₀ values plotted as a function of PRMT3 (●), PRMT4 (∘), PRMT6 (▪) or PRMT8 (□):SAM:Compound A preincubation time.

FIG. 9: MMA in-cell-western. RKO cells were treated with Compound A-tri-HCl (“Compound A-A”), Compound A-mono-HCl (“Compound A-B”), Compound A-free-base (“Compound A-C”), and Compound A-di-HCl (“Compound A-D”) for 72 hours. Cells were fixed, stained with anti-Rme1GG to detect MMA and anti-tubulin to normalize signal, and imaged using the Odyssey imaging system. MMA relative to tubulin was plotted against compound concentration to generate a curve fit (A) in GraphPad using a biphasic curve fit equation. Summary of EC₅₀ (first inflection), standard deviation, and N are shown in (B).

FIG. 10: PRMT1 expression in tumors. mRNA expression levels were obtained from cBioPortal for Cancer Genomics. ACTB levels and TYR are shown to indicate expression of level corresponding to a gene that is ubitiquitously expressed versus one that has restricted expression, respectively.

FIG. 11: Antiproliferative activity of Compound A in cell culture. 196 human cancer cell lines were evaluated for sensitivity to Compound A in a 6-day growth assay. gIC₅₀ values for each cell line are shown as bar graphs with predicted human exposure as indicated in (A). Yₘᵢₙ−T₀, a measure of cytotoxicity, is plotted as a bar-graph in (B), in which gIC₁₀₀ values for each cell line are shown as red dots. The Cₐᵥₑ calculated from the rat 14-day MTD (150 mg/kg, Cₐᵥₑ=2.1 μM) is indicated as a red dashed line.

FIG. 12: Timecourse of Compound A effects on arginine methylation marks in cultured cells. (A) Changes in ADMA, SDMA, and MMA in Toledo DLBCL cells treated with Compound A. Changes in methylation are shown normalized relative to tubulin±SEM (n=3). (B) Representative western blots of arginine methylation marks. Regions quantified are denoted by black bars on the right of the gel.

FIG. 13: Dose response of Compound A on arginine methylation. (A) Representative western blot images of MMA and ADMA from the Compound A dose response in the U2932 cell line. Regions quantified for (B) are denoted by black bars to the left of gels. (B) Minimal effective Compound A concentration required for 50% of maximal induction of MMA or 50% maximal reduction ADMA in 5 lymphoma cell lines after 72 hours of exposure±standard deviation (n=2). Corresponding gIC₅₀ values in 6-day growth death assay are as indicated in red.

FIG. 14: Durability of arginine methylation marks in response to Compound A in lymphoma cells. (A) Stability of changes to ADMA, SDMA, and MMA in the Toledo DLBCL cell line cultured with Compound A. Changes in methylation are shown normalized relative to tubulin±SEM (n=3). (B) Representative western blots of arginine methylation marks. Regions quantified for (A) are denoted by black bars on the side of the gel.

FIG. 15: Proliferation timecourse of lymphoma cell lines. Cell growth was assessed over a 10-day timecourse in the Toledo (A) and Daudi (B) cell lines (n=2 per cell line). Representative data for a single biological replicate are shown.

FIG. 16: Anti-proliferative effects of Compound A in lymphoma cell lines at 6 and 10 days. (A) Average gIC₅₀ values from 6 day (light blue) and 10 day (dark blue) proliferation assays in lymphoma cell lines. (B) Yₘᵢₙ−T₀ at 6 day (light blue) and 10 day (dark blue) with corresponding gIC₁₀₀ (red points).

FIG. 17: Anti-proliferative effects of Compound A in lymphoma cell lines as classified by subtype. (A) gIC₅₀ values for each cell line are shown as bar graphs. Yₘᵢₙ−T₀, a measure of cytotoxicity, is plotted as a bar-graph in (B), in which gIC₁₀₀ values for each cell line are shown as red dots. Subtype information was collected from the ATCC or DSMZ cell line repositories.

FIG. 18: Propidium iodide FACS analysis of cell cycle in human lymphoma cell lines. Three lymphoma cell lines, Toledo (A), U2932 (B), and OCI-Ly1 (C) were treated with 0, 1, 10, 100, 1000, and 10,000 nM Compound A for 10 days with samples taken on days 3, 5, 7, 10 post treatment. Data represents the average±SEM of biological replicates, n=2.

FIG. 19: Caspase-3/7 activation in lymphoma cell lines treated with Compound A. Apoptosis was assessed over a 10-day timecourse in the Toledo (A) and Daudi (B) cell lines. Caspase 3/7 activation is shown as fold-induction relative to DMSO-treated cells. Two independent replicates were performed for each cell line. Representative data are shown for each.

FIG. 20: Efficacy of Compound A in mice bearing Toledo xenografts. Mice were treated QD (37.5, 75, 150, 300, 450, or 600 mg/kg) with Compound A orally or BID with 75 mg/kg (B) over a period of 28 (A) or 24 (B) days and tumor volume was measured twice weekly.

FIG. 21: Effect of Compound A in AML cell lines at 6 and 10 Days. (A) Average gIC₅₀ values from 6 day (light blue) and 10 day (dark blue) proliferation assays in AML cell lines. (B) Yₘᵢₙ−T₀ at 6 day (light blue) and 10 day (dark blue) with corresponding gIC₁₀₀ (red points).

FIG. 22: In vitro proliferation timecourse of ccRCC cines with Compound A. (A) Growth relative to control (DMSO) for 2 ccRCC cell lines. Representative curves from a single replicate are shown. (B) Summary of gIC₅₀ and % growth inhibition for ccRCC cell lines during the timecourse (Average±SD; n=2 for each line).

FIG. 23: Efficacy of Compound A in ACHN xenografts. Mice were treated daily with Compound A orally over a period of 28 days and tumor volume was measured twice weekly.

FIG. 24: Anti-proliferative effects of Compound A in breast cancer cell lines. Bar graphs of gIC₅₀ and growth inhibition (%) (red circles) for breast cancer cell lines profiled with Compound A in the 6-day proliferation assay. Cell lines representing triple negative breast cancer (TNBC) are shown in orange; other subtypes are in blue.

FIG. 25: Effect of Compound A in Breast Cancer Cell Lines at 7 and 12 Days. Average growth inhibition (%) values from 7 day (light blue) and 10 day (dark blue) proliferation assays in breast cancer cell lines with corresponding gIC₅₀ (red points). The increase in potency and percent inhibition observed in long-term proliferation assays with breast cancer, but not lymphoma or AML cell lines, suggest that certain tumor types require a longer exposure to Compound A to fully reveal anti-proliferative activity.

FIG. 26: MTAP status and sensitivity of cancer cell lines to Compound A in culture. Cell lines with deletions of the MTAP locus or downregulation of MTAP RNA were classified as ‘low’ (open circles). Copy number and expression data were downloaded from CCLE.

FIG. 27: Effect of exogenous MTA on potency of Compound A in breast cancer cell lines. EC50, gIC100, Ymin−T0 from 6-day proliferation assays using Compound A and fixed concentrations of MTA. MTAP status is shown above. ND-insufficient growth window with this concentration of MTA to determine parameters.

FIG. 28: Increases in potency of Compound A combined with exogenous MTA. Light gray highlight indicates >5 fold potency increase and dark gray indicates >10 fold. ND-insufficient growth window with this concentration of MTA to determine parameters.

### DETAILED DESCRIPTION OF THE INVENTION

As used herein “Type I protein arginine methyltransferase inhibitor” or “Type I PRMT inhibitor” means an agent that inhibits any one or more of the following: protein arginine methyltransferase 1 (PRMT1), protein arginine methyltransferase 3 (PRMT3), protein arginine methyltransferase 4 (PRMT4), protein arginine methyltransferase 6 (PRMT6) inhibitor, and protein arginine methyltransferase 8 (PRMT8). In some embodiments, the Type I PRMT inhibitor is a small molecule compound. In some embodiments, the Type I PRMT inhibitor selectively inhibits any one or more of the following: protein arginine methyltransferase 1 (PRMT1), protein arginine methyltransferase 3 (PRMT3), protein arginine methyltransferase 4 (PRMT4), protein arginine methyltransferase 6 (PRMT6) inhibitor, and protein arginine methyltransferase 8 (PRMT8). In some embodiments, the Type I PRMT inhibitor is a selective inhibitor of PRMT1, PRMT3, PRMT4, PRMT6, and PRMT8.

Arginine methyltransferases are attractive targets for modulation given their role in the regulation of diverse biological processes. It has now been found that compounds described herein, and pharmaceutically acceptable salts and compositions thereof, are effective as inhibitors or arginine methyltransferases.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3 Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers. Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the namin of any compound described herein does not exclude any tautomer form.

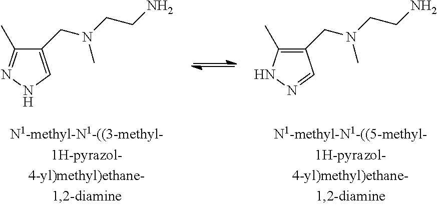

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and subrange within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Radical” refers to a point of attachment on a particular group. Radical includes divalent radicals of a particular group.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆alkyl groups include methyl (C₁, ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms and one or more carbon-carbon double bonds (e.g., 1, 2, 3, or 4 double bonds), and optionally one or more triple bonds (e.g., 1, 2, 3, or 4 triple bonds) (“C₂₋₂₀ alkenyl”). In certain embodiments, alkenyl does not comprise triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”) In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms and one or more carbon-carbon triple bonds (e.g., 1, 2, 3, or 4 triple bonds), and optionally one or more double bonds (e.g., 1, 2, 3, or 4 double bonds) (“C₂₋₂₀ alkynyl”). In certain embodiments, alkynyl does not comprise double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Fused” or “ortho-fused” are used interchangeably herein, and refer to two rings that have two atoms and one bond in common, e.g.,

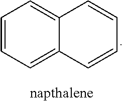

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

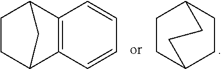

“Spiro” or “Spiro-fused” refers to a group of atoms which connect to the same atom of a carbocyclic or heterocyclic ring system (geminal attachment), thereby forming a ring, e.g.,

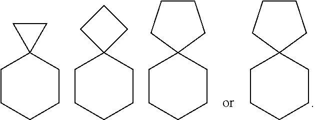

Spiro-fusion at a bridgehead atom is also contemplated.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group refers to a radical of a non-aromatic cyclic hydrocarbon group having from 3 to 10 ring carbon atoms (C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In certain embodiments, heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms independently selected from nitrogen, oxygen, and sulfur. In some embodiments, the 5-6 membered heterocyclyl has 1-2 ring heteroatoms independently selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-14 membered monocyclic or polycyclic (e.g., bicyclic or tricyclic) 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-14 membered aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms independently selected from nitrogen, oxygen, and sulfur. In some embodiments, the 5-6 membered heteroaryl has 1-2 ring heteroatoms independently selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary 5,6-bicyclic heteroaryl groups include, without limitation, any one of the following formulae:

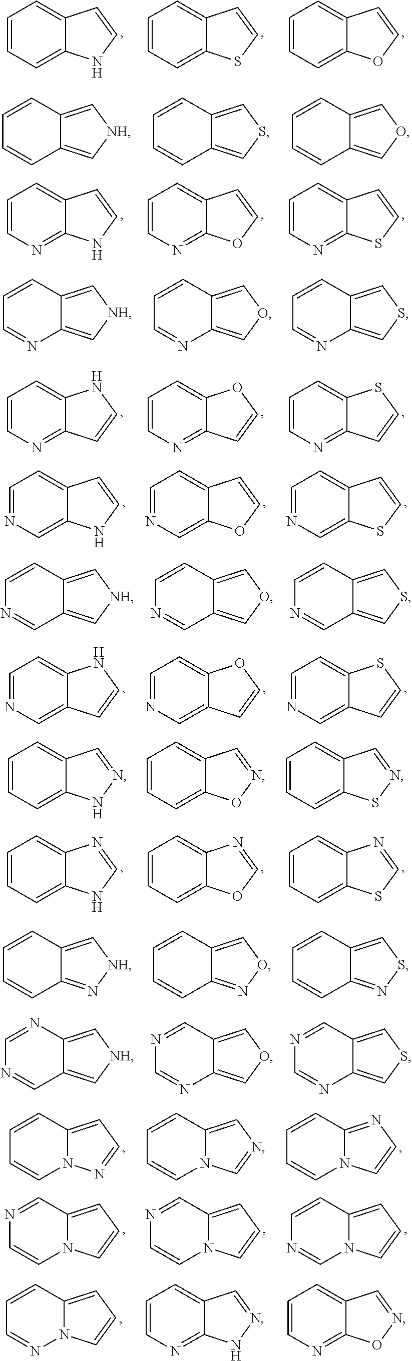

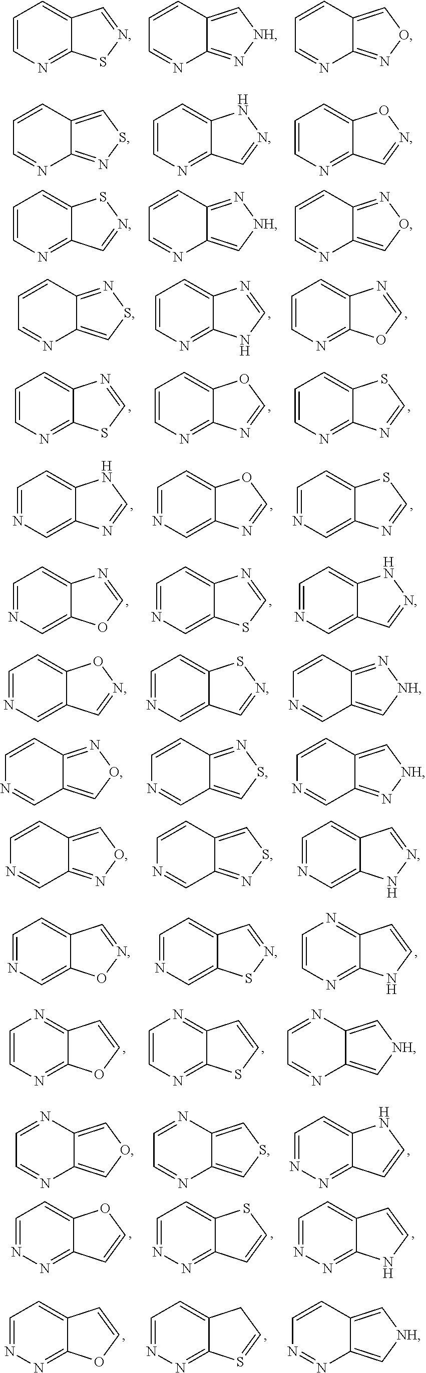

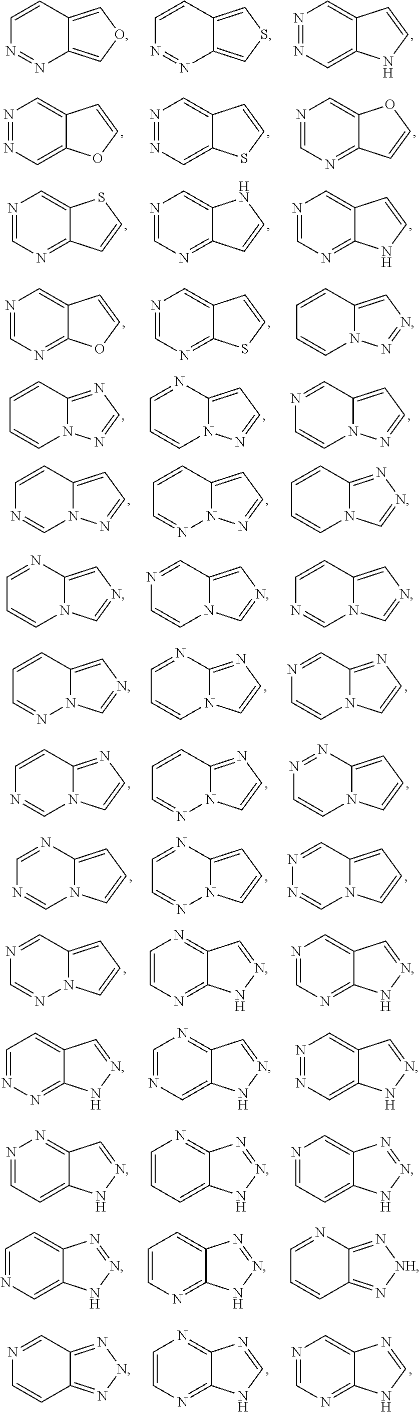

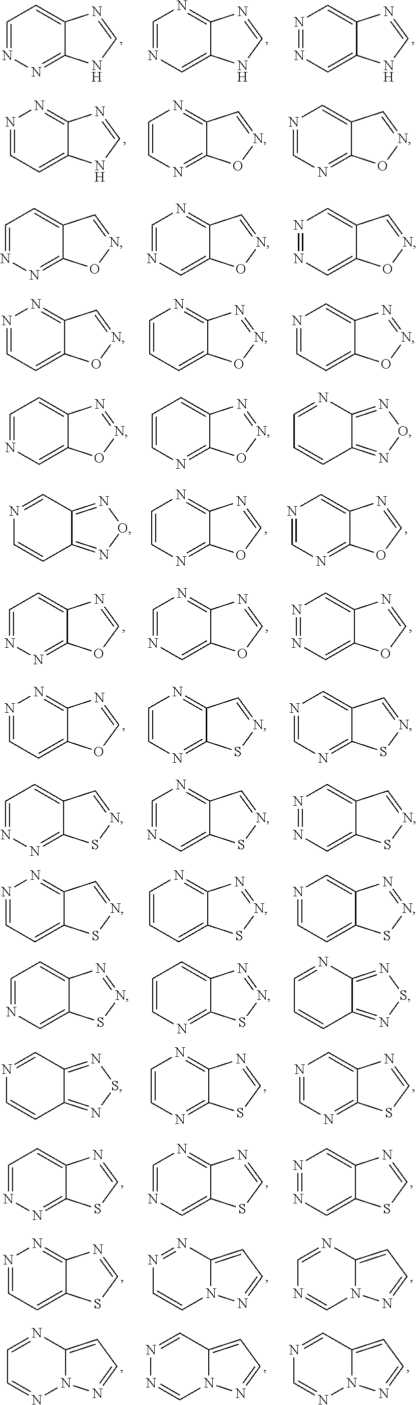

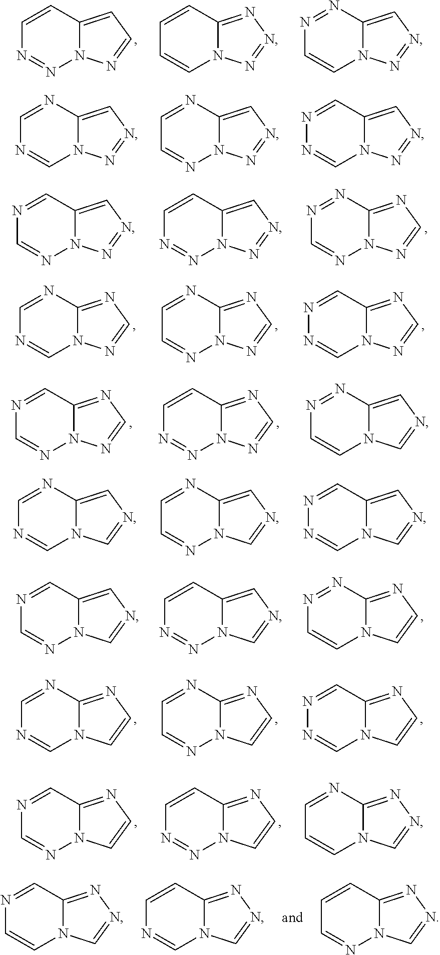

In any of the monocyclic or bicyclic heteroaryl groups, the point of attachment can be any carbon or nitrogen atom, as valency permits.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{CC}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃— —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₁₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —N0₂, —N₃, —SO₂H, —SO₃H, —OH, —OR^{ee}, —ON(R^{ff})₂, —N(R^{ff})₂, —N(R^{ff})₃⁺X, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R)₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR)N(R^{ff})₂, —OC(═NR)N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R)₂, —C(═O)SR^{ee}, —C(═S)SR^{ee}, —SC(═S)SR^{ee}, —P(═O)₂R^{ee}, P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups;

and each instance of R^{gg} is, independently, halogen, —CN, —N0₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻ —NH(C₁₋₆ alkyl)₂⁺X⁻ —NH₂(C₁₋₆ alkyl)⁺X⁻ —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆, alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂ C₁₋₆ alkyl, —SO₂OC₁₋₆ alkyl, —OSO₂ C₁₋₆ alkyl, —SO C₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)S C₁₋₆ alkyl, —P(═O)₂(C₁₋₆ alkyl), —P(═O)(C₁₋₆ alkyl)₂, —OP(═O)(C₁₋₆ alkyl)₂, —OP(═O)(O C₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, CI⁻, Br⁻, I⁻), N0₃⁻, CIO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —CI), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3 rd edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10, 10,10,10-tetrahydrothioxanthyl)] methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)] methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Other nitrogen protecting groups include, but are not limited to, phenothiazinyl-(10)-acyl derivative, N′-p-toluenesulfonylaminoacyl derivative, N-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl] amine (MMTr), N-9-phenylfluorenylamine (PhF), N-2,7-dichloro-9-fluorenylmethyleneamine, N-ferrocenylmethylamino (Fcm), N-2-picolylamino N-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl] amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, Si(R^{aa})₃ —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3 edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1, 1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl,t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO2R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, Si(R^{aa})₃ —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66: 1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The present invention provides Type I PRMT inhibitors. In one embodiment, the Type I PRMT inhibitor is a compound of Formula (I):

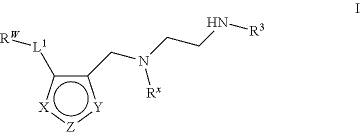

or a pharmaceutically acceptable salt thereof,
wherein

X is N, Z is NR⁴, and Y is CR⁵; or

X is NR⁴, Z is N, and Y is CR⁵; or

X is CR⁵, Z is NR⁴, and Y is N; or

X is CR⁵, Z is N, and Y is NR⁴;

R^{X} is optionally substituted C₁₋₄ alkyl or optionally substituted C₃₋₄ cycloalkyl;

L₁ is a bond, —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, —SO₂N(R^{B})—, or an optionally substituted C₁₋₆ saturated or unsaturated hydrocarbon chain, wherein one or more methylene units of the hydrocarbon chain is optionally and independently replaced with —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—,

—C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, or —SO₂N(R^{B})—;

each R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom;

each R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or an R^{B} and R^{W} on the same nitrogen atom may be taken together with the intervening nitrogen to form an optionally substituted heterocyclic ring;

R^{W} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; provided that when L₁ is a bond, R^{W} is not hydrogen, optionally substituted aryl, or optionally substituted heteroaryl;

R³ is hydrogen, C₁₋₄ alkyl, or C₃₋₄ cycloalkyl;

R⁴ is hydrogen, optionally substituted C₁₋₆ alkyl, optionally substituted C₂₋₆ alkenyl, optionally substituted C₂₋₆ alkynyl, optionally substituted C₃₋₇ cycloalkyl, optionally substituted 4- to 7-membered heterocyclyl; or optionally substituted C₁₋₄ alkyl-Cy;

Cy is optionally substituted C₃₋₇ cycloalkyl, optionally substituted 4- to 7-membered heterocyclyl, optionally substituted aryl, or optionally substituted heteroaryl; and

R⁵ is hydrogen, halo, —CN, optionally substituted C₁₋₄ alkyl, or optionally substituted C₃₋₄ cycloalkyl. In one aspect, R³ is a C₁₋₄ alkyl. In one aspect, R³ is methyl. In one aspect, R⁴ is hydrogen. In one aspect, R⁵ is hydrogen. In one aspect, L₁ is a bond.

In one embodiment, the Type I PRMT inhibitor is a compound of Formula (I) wherein -L₁-R^{W} is optionally substituted carbocyclyl.

In one embodiment, the Type I PRMT inhibitor is a compound of Formula (V)

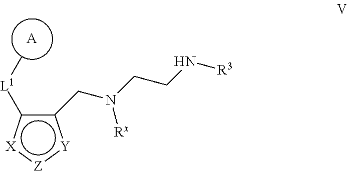

or a pharmaceutically acceptable salt thereof, wherein Ring A is optionally substituted carbocyclyl, optionally substituted heterocyclyl, optionally substituted aryl, or optionally substituted heteroaryl. In one aspect, Ring A is optionally substituted carbocyclyl. In one aspect, R³ is a C₁₋₄ alkyl. In one aspect, R³ is methyl. In one aspect, R^{X} is unsubstituted C₁₋₄ alkyl. In one aspect, R^{X} is methyl. In one aspect, L₁ is a bond.

In one embodiment, the Type I PRMT inhibitor is a compound of Formula (VI)

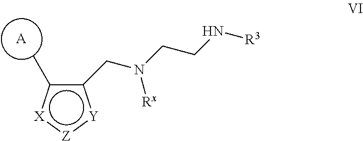

or a pharmaceutically acceptable salt thereof. In one aspect, Ring A is optionally substituted carbocyclyl. In one aspect, R³ is a C₁₋₄ alkyl. In one aspect, R³ is methyl. In one aspect, R^{X} is unsubstituted C₁₋₄ alkyl. In one aspect, R^{X} is methyl.

In one embodiment, the Type I PRMT inhibitor is a compound of Formula (II):

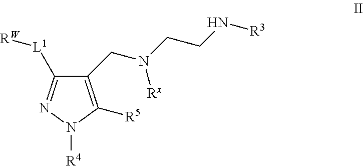

or a pharmaceutically acceptable salt thereof. In one aspect, -L₁-R^{W} is optionally substituted carbocyclyl. In one aspect, R³ is a C₁₋₄alkyl. In one aspect, R³ is methyl. In one aspect, R^{X} is unsubstituted C₁₋₄ alkyl. In one aspect, R^{X} is methyl. In one aspect, R⁴ is hydrogen.

In one embodiment, the Type I PRMT inhibitor is Compound A:

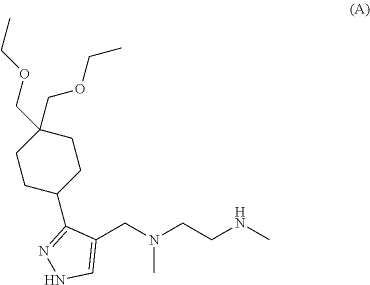 [SMILES: CCOCC1(COCC)CCC(c2n[nH]cc2CN(C)CCNC)CC1]

or a pharmaceutically acceptable salt thereof. Compound A and methods of making Compound A are disclosed in PCT/US2014/029710, in at least page 171 (Compound 158) and page 266, paragraph [00331].

In one embodiment, the Type I PRMT inhibitor is Compound A-tri-HCl, a tri-HCl salt form of Compound A. In another embodiment, the Type I PRMT inhibitor is Compound A-mono-HCl, a mono-HCl salt form of Compound A. In yet another embodiment, the Type I PRMT inhibitor is Compound A-free-base, a free base form of Compound A. In still another embodiment, the Type I PRMT inhibitor is Compound A-di-HCl, a di-HCl salt form of Compound A.

In one embodiment, the Type I PRMT inhibitor is Compound D:

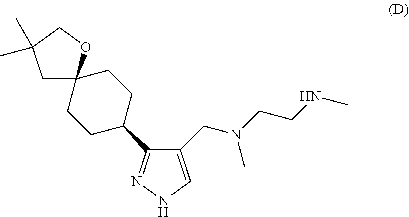

or a pharmaceutically acceptable salt thereof.

Type I PRMT inhibitors are further disclosed in PCT/US2014/029710, which is incorporated herein by reference. Exemplary Type I PRMT inhibitors are disclosed in Table 1A and Table 1B of PCT/US2014/029710, and methods of making the Type I PRMT inhibitors are described in at least page 226, paragraph [00274] to page 328, paragraph [00050] of PCT/US2014/029710.

In one embodiment, methods of treating cancer in a human in need thereof are provided, the methods comprising determining any one or more of: a. the level of 5-Methylthioadenosine phosphorylase (MTAP) polynucleotide or polypeptide, b. the presence or absence of a mutation in MTAP, and c. the level of methylthioadenosine (MTA) in a sample from the human, and administering to the human an effective amount of a Type I protein arginine methyltransferase (Type I PRMT) inhibitor if the level of the MTAP polynucleotide or polypeptide is decreased relative to a control and/or the level of methylthioadenosine (MTA) is increased relative to a control and/or a mutation in MTAP polynucleotide or polypeptide is present, thereby treating the cancer in the human. In one aspect, mutation is an MTAP deletion. In one aspect, the sample comprises a cancer cell. In another aspect, both a and b are determined. In one aspect, the methods further comprise administering one or more additional anti-neoplastic agents. In another aspect, the cancer is a solid tumor or hematological cancer. In one aspect, cancer is lymphoma, acute myeloid leukemia (AML), kidney, melanoma, breast, bladder, colon, lung, or prostate. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, methods of treating cancer in a human in need thereof are provided, the methods comprising determining any one or more of: a. the level of 5-Methylthioadenosine phosphorylase (MTAP) polynucleotide or polypeptide, b. the presence or absence of a mutation in MTAP, and c. the level of methylthioadenosine (MTA) in a sample from the human, and administering to the human an effective amount of Compound A if the level of the MTAP polynucleotide or polypeptide is decreased relative to a control and/or the level of methylthioadenosine (MTA) is increased relative to a control and/or a mutation in MTAP polynucleotide or polypeptide is present, thereby treating the cancer in the human. In another embodiment, methods of treating cancer in a human in need thereof are provided, the methods comprising determining a. the level of 5-Methylthioadenosine phosphorylase (MTAP) polynucleotide or polypeptide, or b. the presence or absence of a mutation in MTAP in a sample from the human, and administering to the human an effective amount of Compound A if the level of the MTAP polynucleotide or polypeptide is decreased relative to a control or a mutation in MTAP polynucleotide or polypeptide is present, thereby treating the cancer in the human. In some aspects, the level of MTAP polynucleotide or polypeptide is decreased by at least about 10%, at least about 20%, at least about 30%, at least about 40%, at least about 50%, at least about 60%, at least about 70%, at least about 80%, at least about 90%, at least about 95%, or at least about 99% relative to the control. In some other aspects, the level of MTA is increased by at least about 2-fold, at least about 3-fold, at least about 4-fold, at least about 5-fold, at least about 10-fold, at least about 15-fold, at least about 20-fold, at least about 25-fold, 30-fold, at least about 35-fold, at least about 40-fold, at least about 45-fold, or at least about 50-fold relative to the control.

In another embodiment, methods of inhibiting proliferation of a cancer cell in a human in need thereof are provided, the methods comprising administering to the human an effective amount of a Type I protein arginine methyltransferase (Type I PRMT) inhibitor, thereby inhibiting proliferation of the cancer cell in the human, wherein the cancer cell has a mutation in 5-Methylthioadenosine phosphorylase (MTAP) and/or a decreased level of a MTAP polynucleotide or polypeptide relative to a control and/or an increased level of methylthioadenosine (MTA) relative to a control. In one aspect, the mutation is an MTAP deletion. In one aspect, the decreased level of MTAP polynucleotide or polypeptide or the mutation in MTAP increases the level of methylthioadenosine (MTA) in the cancer cell such that the activity of protein arginine methyltransferase 5 (PRMT5) is inhibited. In one aspect, the decreased level of MTAP polynucleotide or polypeptide or the mutation in MTAP in the cancer cell increases sensitivity of the cancer cell to the Type I PRMT inhibitor. In one aspect, the cancer cell is a solid tumor cancer cell or hematological cancer cell. In another aspect, the cancer cell is a lymphoma cell, acute myeloid leukemia (AML) cell, kidney cancer cell, melanoma cell, breast cancer cell, bladder cancer cell, colon cancer cell, lung cancer cell, or prostate cancer cell. In one aspect, the Type I PRMT inhibitor is a compound of Formula I, II, V, or VI. In one aspect, the Type I PRMT inhibitor is Compound A. In another aspect, the Type I PRMT inhibitor is Compound D. In another embodiment, methods of inhibiting proliferation of a cancer cell in a human in need thereof are provided, the methods comprising administering to the human an effective amount of Compound A, thereby inhibiting proliferation of the cancer cell in the human, wherein the cancer cell has a mutation in 5-Methylthioadenosine phosphorylase (MTAP) and/or a decreased level of a MTAP polynucleotide or polypeptide relative to a control and/or an increased level of methylthioadenosine (MTA) relative to a control. In some aspects, the level of MTAP polynucleotide or polypeptide is decreased by at least about 10%, at least about 20%, at least about 30%, at least about 40%, at least about 50%, at least about 60%, at least about 70%, at least about 80%, at least about 90%, at least about 95%, or at least about 99% relative to the control. In some other aspects, the level of MTA is increased by at least about 2-fold, at least about 3-fold, at least about 4-fold, at least about 5-fold, at least about 10-fold, at least about 15-fold, at least about 20-fold, at least about 25-fold, 30-fold, at least about 35-fold, at least about 40-fold, at least about 45-fold, or at least about 50-fold relative to the control.

In yet another embodiment, the present invention provides methods of predicting whether a human having cancer will be sensitive to treatment with a Type I protein arginine methyltransferase (Type I PRMT) inhibitor, the methods comprising determining a. the level of 5-Methylthioadenosine phosphorylase (MTAP) polynucleotide or polypeptide or b. the presence or absence of a mutation in MTAP in a sample from the human, wherein a decreased level of MTAP polynucleotide or polypeptide relative to a control or the presence of a mutation in MTAP indicates the human will be sensitive to treatment with a Type I PRMT inhibitor. In another embodiment, the present invention provides methods of predicting whether a human having cancer will be sensitive to treatment with a Type I protein arginine methyltransferase (Type I PRMT) inhibitor, the methods comprising determining any one or more of: a. the level of 5-Methylthioadenosine phosphorylase (MTAP) polynucleotide or polypeptide, b. the presence or absence of a mutation in MTAP, and c. the level of methylthioadenosine (MTA) in a sample from the human, wherein a decreased level of MTAP polynucleotide or polypeptide relative to a control and/or the presence of a mutation in MTAP and/or an increased level of MTA relative to a control indicates the human will be sensitive to treatment with a Type I PRMT inhibitor. In one aspect, mutation is an MTAP deletion. In one aspect, the sample comprises a cancer cell. In one aspect, both a and b are determined. In another aspect, the methods further comprise administering one or more additional anti-neoplastic agents. In one aspect, the cancer is a solid tumor or hematological cancer. In one aspect, cancer is lymphoma, acute myeloid leukemia (AML), kidney, melanoma, breast, bladder, colon, lung, or prostate. In one aspect, the Type I PRMT inhibitor is a compound of Formula I, II, V, or VI. In one aspect, the Type I PRMT inhibitor is Compound A. In another aspect, the Type I PRMT inhibitor is Compound D In some aspects, the level of MTAP polynucleotide or polypeptide is decreased by at least about 10%, at least about 20%, at least about 30%, at least about 40%, at least about 50%, at least about 60%, at least about 70%, at least about 80%, at least about 90%, at least about 95%, or at least about 99% relative to the control. In some other aspects, the level of MTA is increased by at least about 2-fold, at least about 3-fold, at least about 4-fold, at least about 5-fold, at least about 10-fold, at least about 15-fold, at least about 20-fold, at least about 25-fold, 30-fold, at least about 35-fold, at least about 40-fold, at least about 45-fold, or at least about 50-fold relative to the control.

In another embodiment, a Type I PRMT inhibitor for use in the treatment of cancer in a human classified as a responder is provided, wherein a responder is characterized by the presence of a mutation in 5-Methylthioadenosine phosphorylase (MTAP) or a decreased level of MTAP polynucleotide or polypeptide relative to a control or an increased level of methylthioadenosine (MTA) relative to a control in a sample from the human. In one aspect, mutation is an MTAP deletion. In one aspect, the sample comprises a cancer cell. In one aspect, the responder is characterized by the presence of a mutation in 5-Methylthioadenosine phosphorylase (MTAP). In another aspect, the responder is characterized by the presence of a mutation in 5-Methylthioadenosine phosphorylase (MTAP) and a decreased level of MTAP polynucleotide or polypeptide relative to a control. In still another aspect, the responder is characterized by the presence of a mutation in 5-Methylthioadenosine phosphorylase (MTAP), a decreased level of MTAP polynucleotide or polypeptide relative to a control, and an increased level of methylthioadenosine (MTA) relative to a control in a sample from the human. In some aspects, the level of MTAP polynucleotide or polypeptide is decreased by at least about 10%, at least about 20%, at least about 30%, at least about 40%, at least about 50%, at least about 60%, at least about 70%, at least about 80%, at least about 90%, at least about 95%, or at least about 99% relative to the control. In some other aspects, the level of MTA is increased by at least about 2-fold, at least about 3-fold, at least about 4-fold, at least about 5-fold, at least about 10-fold, at least about 15-fold, at least about 20-fold, at least about 25-fold, 30-fold, at least about 35-fold, at least about 40-fold, at least about 45-fold, or at least about 50-fold relative to the control. In another aspect, the methods further comprise administering one or more additional anti-neoplastic agents. In one aspect, the cancer is a solid tumor or hematological cancer. In one aspect, cancer is lymphoma, acute myeloid leukemia (AML), kidney, melanoma, breast, bladder, colon, lung, or prostate. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, Compound A for use in the treatment of cancer in a human classified as a responder is provided, wherein a responder is characterized by the presence of a mutation in 5-Methylthioadenosine phosphorylase (MTAP) or a decreased level of MTAP polynucleotide or polypeptide relative to a control or an increased level of methylthioadenosine (MTA) relative to a control in a sample from the human. In one embodiment, Compound A for use in the treatment of cancer in a human classified as a responder is provided, wherein a responder is characterized by the presence of an MTAP deletion in a sample from the human.

In another embodiment, the present invention provides a mutation in 5-Methylthioadenosine phosphorylase (MTAP) for use as a biomarker in the treatment/diagnosis of a cancer responsive to a Type I PRMT inhibitor. In one embodiment, the present invention provides an MTAP deletion mutation for use as a biomarker in the treatment/diagnosis of a cancer responsive to a Type I PRMT inhibitor. In another embodiment, the present invention provides a mutation in 5-Methylthioadenosine phosphorylase (MTAP) for use as a biomarker in the treatment/diagnosis of a cancer responsive to Compound A. In one embodiment, the present invention provides an MTAP deletion mutation for use as a biomarker in the treatment/diagnosis of a cancer responsive to Compound A.

In another embodiment, the present invention provides a mutation in 5-Methylthioadenosine phosphorylase (MTAP) for use in a diagnostic method. In one embodiment, the present invention provides an MTAP deletion mutation for use in a diagnostic method. In another embodiment, the present invention provides a mutation in 5-Methylthioadenosine phosphorylase (MTAP) for use in therapy. In one embodiment, the present invention provides an MTAP deletion mutation for use in therapy.

The terms “polypeptide” and “protein” are used interchangeably and are used herein as a generic term to refer to native protein, fragments, peptides, or analogs of a polypeptide sequence. Hence, native protein, fragments, and analogs are species of the polypeptide genus.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

As used herein, “MTAP” or “5-Methylthioadenosine phosphorylase” is a protein that catalyzes the reversible phosphorylation of methylthioadenosine (MTA) to adenine and 5-methylthioribose-1-phosphate (Accession No.: UniprotKB—Q13126 (MTAP_HUMAN)). The sequence of MTAP as shown in UniprotKB—Q13126-1 (Isoform 1) is reproduced below:

(SEQ ID NO: 1)
10 20 30 40
MASGTTTTAV KIGIIGGTGL DDPEILEGRT EKYVDTPFGK
50 60 70 80
PSDALILGKI KNVDCVLLAR HGRQHTIMPS KVNYQANIWA
90 100 110 120
LKEEGCTHVI VTTACGSLRE EIQPGDIVII DQFIDRTTMR
130 140 150 160
PQSFYDGSHS CARGVCHIPM AEPFCPKTRE VLIETAKKLG
170 180 190 200
LRCHSKGTMV TIEGPRFSSR AESFMFRTWG ADVINMTTVP
210 220 230 240
EVVLAKEAGI CYASIAMATD YDCWKEHEEA VSVDRVLKTL
250 260 270 280
KENANKAKSL LLTTIPQIGS TEWSETLHNL KNMAQFSVLL
PRH.

As used herein, an “MTAP polynucleotide” means a polynucleotide encoding an MTAP polypeptide. An exemplary MTAP polynucleotide sequence can be found in NCBI Reference Sequence: NM_002451.3. The sequence shown in NM_002451.3 is reproduced below:

```
(SEQ ID NO: 2)
      1 ctccgcactg ctcactcccg cgcagtgagg ttggcacagc caccgctctg tggctcgctt
    61 ggttccctta gtcccgagcg ctcgcccact gcagattcct ttcccgtgca gacatggcct
  121 ctggcaccac caccaccgcc gtgaagattg gaataattgg tggaacaggc ctggatgatc
  181 cagaaatttt agaaggaaga actgaaaaat atgtggatac tccatttggc aagccatctg
  241 atgccttaat tttggggaag ataaaaaatg ttgattgcgt cctccttgca aggcatggaa
  301 ggcagcacac catcatgcct tcaaaggtca actaccaggc gaacatctgg gctttgaagg
  361 aagagggctg tacacatgtc atagtgacca cagcttgtgg ctccttgagg gaggagattc
  421 agcccggcga tattgtcatt attgatcagt tcattgacag gaccactatg agacctcagt
  481 ccttctatga tggaagtcat tcttgtgcca gaggagtgtg ccatattcca atggctgagc
  541 cgttttgccc caaaacgaga gaggttctta tagagactgc taagaagcta ggactccggt
  601 gccactcaaa ggggacaatg gtcacaatcg agggacctcg ttttagctcc cgggcagaaa
  661 gcttcatgtt ccgcacctgg ggggcggatg ttatcaacat gaccacagtt ccagaggtgg
  721 ttcttgctaa ggaggctgga atttgttacg caagtatcgc catggcgaca gattatgact
  781 gctggaagga gcacgaggaa gcagtttcgg tggaccgggt cttaaagacc ctgaaagaaa
  841 acgctaataa agccaaaagc ttactgctca ctaccatacc tcagataggg tccacagaat
  901 ggtcagaaac cctccataac ctgaagaata tggcccagtt ttctgtttta ttaccaagac
  961 attaaagtag catggctgcc caggagaaaa gaagacattc taattccagt cattttggga
1021 attcctgctt aacttgaaaa aaatatggga aagacatgca gctttcatgc ccttgcctat
1081 caaagagtat gttgtaagaa agacaagaca ttgtgtgtat tagagactcc tgaatgattt
1141 agacaacttc aaaatacaga agaaaagcaa atgactagta aacatgtggg aaaaaatatt
1201 acattttaag ggggaaaaaa aaacccacca ttctcttctc cccctattaa atttgcaaca
1261 ataaagggtg gagggtaatc tctactttcc tatactgcca aagaatgtga ggaagaaatg
1321 ggactctttg gttatttatt gatgcgactg taaattggta cagtatttct ggagggcaat
1381 ttggtaaaat gcatcaaaag acttaaaaat acggacgtac tttgtgctgg gaactctaca
1441 tctagcaatt tctctttaaa accatatcag agatgcatac aaagaattat atataaagaa
1501 gggtgtttaa taatgatagt tataataata aataattgaa acaatctgaa tcccttgcaa
1561 ttggaggtaa attatgtctt agttataatt agattgtgaa tcagccaact gaaaatcctt
1621 tttgcatatt tcaatgtcct aaaaagacac ggttgctcta tatatgaagt gaaaaaagga
1681 tatggtagca ttttatagta ctagttttgc tttaaaatgc tatgtaaata tacaaaaaaa
1741 ctagaaagaa atatatataa ccttgttatt gtatttgggg gagggatact gggataattt
1801 ttattttctt tgaatctttc tgtgtcttca catttttcta cagtgaattt aatcaaatag
1861 taaagttgtt gtaaaaataa aagtggattt agaaagatcc agttcttgaa aacactgttt
1921 ctggtaatga agcagaattt aagttggtaa tattaaggtg aatgtcattt aagggagtta
1981 catctttatt ctgctaaaga agaggatcat tgatttctgt acagtcagaa cagtacttgg
2041 gtttgcaaca gctttctgag aaaagctagg tgtttaatag tttaactgaa agtttaacta
2101 tttaaaagac taaatgcaca ttttatggta tctgatattt taaaaagtaa tgtttgattc
2161 tcctttttat gagttaaatt attttatacg agttggtaat ttttgctttt taataaagtg
2221 gaagcttgct tttttaactc tttttttatt gttattttat agaaatgctt tttgttggcc
2281 gggcacagtt gctcatccat gtaatcccag cactgtggga ggccgagacg ggtggatcac
2341 aaggtcagga gatcgagacc atcctggcta atgcgttgaa actccgtctc tactaaaaat
2401 acaaaaaatt agctgggcgt ggtggtgggc acctgtagtc ccagctactc aggaggctga
2461 ggcaggagaa tggtgtgaac ctgggaggtg gagcttgcag tgagcagagc ttgcagtgag
2521 acgagcttgt gccactgcac tccagcctgg gcaacagagt aagactcagt ctcaaaaaaa
2581 aaaaaaagag tgaaatgctt tttgtttgct tcagtttttt atcatgggga gatctttttc
2641 ctcagaattg ttttcttttc actgtaggct attacaggat acttcaggat caagatacag
2701 aaccttttat ttaaagagtt tgtaaagtca atgtgtttgt ttgtgtctct gagattgact
2761 tcaagataat aagctgctaa ttgtaaacaa aacagttacc ctccagtatt aatatgactc
2821 attagtgtga gccatttggg tcaagtatga ttatgaccct tggacttcct gatgtagtat
2881 taaatttcaa ctctggttat ccattagcaa tctgtagaga acttaatgaa cctgaaccca
2941 ggcttctcta gctctggtaa cgtgtgattg ttttcactac aatatgatac atagatggta
3001 ccttactttt cctcattctt aataggtgtc taagaatgtc agggcaaaag tatgggcatt
3061 tttcttgcta tgttcagaaa gtacagttct ctccaacttg cagaggtact tttcttgatt
3121 aaatagcctt ctctagcaac atcattttca gactaactaa atgaatgcag tatactcttt
3181 tctttgttct caatcattca ctccttatgc aaagccaata taattttcct cataccttat
3241 gcttgaggat attgttgaag aacacttcct ggaacacttc tcacttgtga tgctgtacta
3301 attttttttt tttaatttaa gctagtatac taagtgaaca ccatggtcag ttgtgagcat
3361 tttggtttcc gcaaaggatg gatggtgagc atcatgggaa agctgtagtt tagtgactta
3421 gcccttagtg attaatagat ttgcatgtac atagaagtct ttgttggcct tataatctgc
3481 tgttatattt ggcatggatt ttcatggttt tgagaatgac atcctggccc tgtggtcccc
3541 gagggtcatg gtccttgtga cctggcccct gttcactgcc cccttcgcta gcacgagttg
3601 ctgtgcaggg ctggaggtag ctaccatggc ttgtttcaag gaaggaaact ctggtacggt
3661 ggcaccctca ggagtggagg acagtgaact tccttgaaga gggagtgact aaggtgacct
3721 ccaacctgcc ctgagccagc tgccctgcag gtgccacgtg agcctgctct ggcatccaca
3781 ggatgctcct ggagcctctt ctctggctgc tacctcaggg catggttgtg gccccaccaa
3841 cacctatttt ccaaataatt attcattctt gtgacagtgg cctgaacatg tttttaattt
3901 tctcaacaag catttagcca gcacttatcc agtgaaacaa tttgataagg tttcaaggag
3961 tatctgatgg gttaggaagt cacgaaatga ggagttcttg ccacatttgc agagtccctc
4021 cttgataagg tttggcggtg tccccaccca aatctcatgt tgaattgtag ttcccataat
4081 ccccacatgt tgtgggaggg acccagtggg aggtaattaa atcatggggg tggttacccc
4141 cacactgctg ttctcatgat actgagttct cacaagtcct gtttgtttta taaggggctt
4201 ttcccccttt tgctcaacac ttcttcctgc catcatgtga agaaggacgt gtttgtttcc
4261 ccttctgcca cgattgtaag tttcctgagg ccttcccagc tatgtggaac tgtgagttaa
4321 ttaaacctct ttcctttata aattacccag tcatgggcag tcctttacag cagcatgaga
4381 atggactaat acactcctca aatgttttga agattgttgc accttggaac taccagtgtg
4441 cacacaatct ggctcaatgt atatattggc ccagcaaggc aaagaactga agttccagga
4501 tggaagaacc tgtgttctcc tcataatagt atagaataat tcaagatagg caagaaggac
4561 agcagtaaat gaagaccatg gaagaaaaga aggaatgcca aagatcgagg aaatctacca
4621 agactagtag ggtagtccag aagaagctgt ttcagggcct gttgccagct atgcctttga
4681 gaacctcggg atcccaaaga atgaggggaa tttcttcaga aagacaatct cggcatgcat
4741 tatttctttg ttttgaagat tcactcatgt tgcatgcatc tgtagcttgt gcctttttta
4801 ttgcctagta gtattctgtc atatgcctat cttacaattt gattatctat tcacctgttg
4861 atgaatgttt gaattttttc catttgagga attttatgaa taaagctgct ataagcatga
4921 aaaaaaaaaa aaaaaaa.
```

By “methylthioadenosine” or “MTA” or “5-methylthioadenosine” is meant a compound having a structure as shown below:

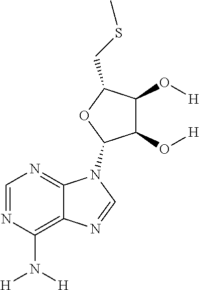 [SMILES: CSC[C@H]1O[C@@H](n2cnc3c(N)ncnc32)[C@H](O)[C@@H]1O]

Levels of MTA in a sample can be measured by a number of methods well known in the art. For example, MTA levels in a sample can be measured using liquid chromatography-mass spectrometry (LC-MS). Measurement of MTA levels using LC-MS is described in, for example, Mavrakis, K. J. et al., Disordered methionine metabolism in MTAP/CDKN2A-deleted cancers leads to dependence on PRMT5. Science 351, 1208-1213, doi:10.1126/science.aad5944 (2016).

A “mutation” in a polypeptide or a gene encoding a polypeptide and grammatical variations thereof means a polypeptide or gene encoding a polypeptide having one or more allelic variants, splice variants, derivative variants, substitution variants, deletion variants, and/or insertion variants, fusion polypeptides, orthologs, and/or interspecies homologs. By way of example, at least one mutation of MTAP would include an MTAP in which part of all of the sequence of a polypeptide or polynucleotide encoding the polypeptide is absent or not expressed in the cell for at least one of the MTAP proteins produced in the cell. For example, an MTAP protein may be produced by a cell in a truncated form and the sequence of the truncated form may be wild type over the sequence of the truncate. A deletion may mean the absence of all or part of a gene or protein encoded by a gene. An MTAP mutation also means a mutation at a single base in a polynucleotide, or a single amino acid substitution. Additionally, some of a protein expressed in or encoded by a cell may be mutated, e.g., at a single amino acid, while other copies of the same protein produced in the same cell may be wild type.

Mutations may be detected in the polynucleotide or translated protein by a number of methods well known in the art. These methods include, but are not limited to, sequencing, RT-PCR, and in situ hybridization, such as fluorescence-based in situ hybridization (FISH), antibody detection, protein degradation sequencing, etc. Methods of detecting a mutation in MTAP, e.g. an MTAP deletion, are well known to one of skill in the art and are described herein in the detailed description and Examples. Methods of determining a decreased level of MTAP polynucleotide or polypeptide are well known in the art and shown in the Examples. The methods can include using primers specific for MTAP polynucleotide or an antibody specific for MTAP polypeptide.

Samples, e.g. biological samples, for testing or determining of one or more mutations may be selected from the group of proteins, nucleotides, cellular blebs or components, serum, cells, blood, blood components, urine and saliva. Testing for mutations may be conducted by several techniques known in the art and/or described herein. In some embodiments, the sample contains one or more cancer cells.

A control can be any one of skill in the art would choose, such as a matched cell from a human, a matched tissue from a human, a cell of the same origin as the tumor but known to have wild type MTAP, or a devised control that correlates with what is seen in non-cancerous cells of the same origin or in cells with wild-type MTAP.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature 5 Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

In one embodiment, a kit for the treatment of cancer is provided, comprising a kit for determining one or more of a and b of claim 1, and a means for determining one or more of a or b of claim 1. In one aspect, the means is selected from the group consisting of primers, probes, and antibodies.

An oligonucleotide probe, or probe, is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions.

The term “oligonucleotide” referred to herein includes naturally occurring and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes, although oligonucleotides may be double stranded, e.g. for use in the construction of a gene mutant. Oligonucleotides can be either sense or antisense oligonucleotides.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

In one embodiment, the present invention provides a pharmaceutical composition comprising a Type I PRMT inhibitor or a pharmaceutically acceptable salt thereof, for use in treating cancer in a human wherein at least a first sample from the human is determined to have a mutation in MTAP, a decreased level of level of MTAP polynucleotide or polypeptide relative to a control, or both.

In one embodiment, use of a Type I PRMT inhibitor in the manufacture of a medicament for the treatment of cancer in a human is provided, wherein one or more samples from the human is determined to have a mutation in MTAP, a decreased level of MTAP polynucleotide or polypeptide relative to a control, or both.

In one aspect the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

In one aspect, the methods of the present invention further comprise administering administering one or more additional anti-neoplastic agents to the human.

In one aspect the human has a solid tumor. In one aspect the tumor is selected from head and neck cancer, gastric cancer, melanoma, renal cell carcinoma (RCC), esophageal cancer, non-small cell lung carcinoma, prostate cancer, colorectal cancer, ovarian cancer and pancreatic cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10^{th} edition (Dec. 5, 2014), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents such as diterpenoids and vinca alkaloids; platinum coordination complexes; alkylating agents such as nitrogen mustards, oxazaphosphorines, alkylsulfonates, nitrosoureas, and triazenes; antibiotic agents such as actinomycins, anthracyclins, and bleomycins; topoisomerase I inhibitors such as camptothecins; topoisomerase II inhibitors such as epipodophyllotoxins; antimetabolites such as purine and pyrimidine analogues and anti-folate compounds; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents such as genetically modified T cells.

Examples of a further active ingredient or ingredients for use in combination or co-administered with the present methods or combinations are anti-neoplastic agents. Examples of anti-neoplastic agents include, but are not limited to, chemotherapeutic agents; immuno-modulatory agents; immune-modulators; and immunostimulatory adjuvants.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7,10 1,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani M. C., et al., J. Am. Chem. Soc., 93(9): 2325-2327 (1971), who characterized its structure by chemical and X-ray crystallographic methods. One mechanism for its activity relates to paclitaxel's capacity to bind tubulin, thereby inhibiting cancer cell growth (Schiff P. B. and Horwitz S. B., Proc. Natl. Acad. Sci. USA, 77: 1561-1565 (1980); Schiff P. B., et al., Nature, 277: 665-667 (1979); Kumar N., J. Biol. Chem., 256: 10435-10441 (1981)). For a review of synthesis and anticancer activity of some paclitaxel derivatives see: D. G. I. Kingston et al., Studies in Organic Chemistry vol. 26, entitled “New Trends in Natural Products Chemistry 1986”, Atta-ur-Rahman, P. W. Le Quesne, Eds. (Elsevier, Amsterdam, 1986) pp 219-235.

Paclitaxel has been approved for clinical use for the treatment of refractory ovarian cancer in the United States (Markman M., Yale J. Biol. Med., 64(6): 583-590 (1991); McGuire W. P., et al., Ann. Intern. Med., 111(4): 273-279 (1989)) and for the treatment of breast cancer (Holmes F. A., et al., J. Natl. Cancer Inst., 83(24): 1797-1805 (1991)). It is a potential candidate for treatment of neoplasms in the skin (Einzig A. I., et. al., Cancer Treat. Res., 58: 89-100 (1991)) and head and neck carcinomas (Forastiere A. A., Semin. Oncol., 20(4 Suppl. 3): 56-60 (1993). The compound also shows potential for the treatment of polycystic kidney disease (Woo D. D., et. al., Nature, 368(6473): 750-753 (1994)), lung cancer and malaria. Treatment of patients with paclitaxel results in bone marrow suppression (Ignoffo R. J. et. al, Cancer Chemotherapy Pocket Guide, (1998)) related to the duration of dosing above a threshold concentration (50 nM) (Kearns, C. M., et. al., Semin. Oncol., 22(3 Suppl. 6): 16-23 (1995)).

Docetaxel, (2R,3S)—N-carboxy-3-phenylisoserine,N-tert-butyl ester, 13-ester with 5β-20-epoxy-1,2α,4,7,10β,13α-hexahydroxytax-11l-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel, prepared using a natural precursor, 10-deacetyl-baccatin III, extracted from the needle of the European Yew tree. The main dose limiting toxicity of docetaxel treatment is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although it has possible indications as a second line therapy of various solid tumors, it is primarily indicated for the treatment of testicular cancer and various lymphomas including Hodgkin's disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effects of vincristine and to a lesser extent myelosupression and gastrointestinal mucositis effects occur.

Vinorelbine, 3′,4′-didehydro-4‘-deoxy-C’-norvincaleukoblastine [R—(R*,R*)-2,3-dihydroxybutanedioate (1:2)salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, for the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo aquation, and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated for the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)—O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, for the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's disease. Nausea, vomiting, and anorexia are the most common dose limiting side effects of dacarbazine.

Antibiotic anti-neoplastics are non-phase specific agents, which bind or intercalate with DNA. This action disrupts the ordinary function of the nucleic acids, leading to cell death. Examples of antibiotic anti-neoplastic agents include, but are not limited to, actinomycins such as dactinomycin; anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-a-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction for the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-a-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component for the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase I inhibitors include, but are not limited to, camptothecins. The cytotoxic activity of camptothecins is believed to be related to its topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin.

Irinotecan, (4S)-4,11-diethyl-4-hydroxy-9-[(4-piperidinopiperidino) carbonyloxy]-1H-pyrano[3′,4′,6,7]indolizino[1,2-b]quinoline-3,14(4H, 12H)-dione hydrochloride, is commercially available as the injectable solution CAMPTOSAR®. Irinotecan is a derivative of camptothecin, which binds, along with its active metabolite SN-38, to the topoisomerase I—DNA complex. It is believed that cytotoxicity occurs as a result of irreparable double strand breaks caused by interaction of the topoisomerase I: DNA: irinotecan or SN-38 ternary complex with replication enzymes. Irinotecan is indicated for treatment of metastatic cancer of the colon or rectum. The dose limiting side effects of irinotecan are myelosuppression, including neutropenia, and GI effects, including diarrhea.

Topotecan, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I—DNA complex and prevents religation of singles strand breaks caused by topoisomerase I in response to torsional strain of the DNA molecule. Topotecan is indicated for second line treatment of metastatic carcinoma of the ovary and small cell lung cancer. The dose limiting side effect of topotecan is myelosuppression, primarily neutropenia.

Also of interest, is the camptothecin derivative of formula A′ following, currently under development, including the racemic mixture (R,S) form as well as the R and S enantiomers:

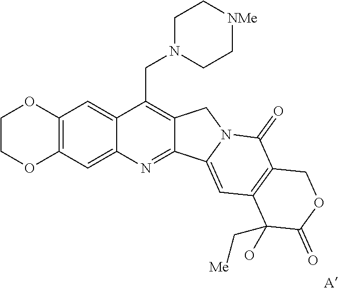 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound, as well as related compounds, is described, including methods of making, in U.S. Pat. Nos. 6,100,273, 6,063,923; 5,342,947; 5,559,235; and 5,491,237.
Topoisomerase II inhibitors include, but are not limited to, epipodophyllotoxins. Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents for the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents for the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents for the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dihydrofolic acid reductase which is required for synthesis of purine nucleotides and thymidylate. Methotrexate is indicated as a single agent or in combination with other chemotherapy agents for the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effects of methotrexate administration.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents for the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents for the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents for the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′, 2′-difluorocytidine monohydrochloride (p-isomer), is commercially available as GEMZAR®. Gemcitabine exhibits cell phase specificity at S-phase and by blocking progression of cells through the G1/S boundary. Gemcitabine is indicated in combination with cisplatin for the treatment of locally advanced non-small cell lung cancer and alone for the treatment of locally advanced pancreatic cancer. Myelosuppression, including leucopenia, thrombocytopenia, and anemia, is the most common dose limiting side effect of gemcitabine administration.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone, which are useful for the treatment of malignant lymphoma and acute leukemia in children; aminoglutethimide and other aromatase inhibitors such as anastrozole, letrazole, vorazole, and exemestane, which are useful for the treatment of adrenocortical carcinoma and hormone dependent breast carcinoma containing estrogen receptors; progestrins such as megestrol acetate, which are useful for the treatment of hormone dependent breast cancer and endometrial carcinoma; estrogens, androgens, and anti-androgens such as flutamide, nilutamide, bicalutamide, cyproterone acetate and Sa-reductases such as finasteride and dutasteride, which are useful for the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, which are useful for the treatment of hormone dependent breast carcinoma and other susceptible cancers; and gonadotropin-releasing hormone (GnRH) and analogues thereof, which stimulate the release of leutinizing hormone (LH) and/or follicle stimulating hormone (FSH) for the treatment prostatic carcinoma, for instance, LHRH agonists and antagonists such as goserelin acetate and luprolide.

Signal transduction pathway inhibitors are those inhibitors, which block or inhibit a chemical process which evokes an intracellular change. As used herein, this change is cell proliferation or differentiation. Signal transduction inhibitors useful in the present invention include, but are not limited to, inhibitors of receptor tyrosine kinases, non-receptor tyrosine kinases, SH2/SH3domain blockers, serine/threonine kinases, phosphatidyl inositol-3 kinases, myo-inositol signalling, and Ras oncogenes.

Several protein tyrosine kinases catalyze the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFR), tyrosine kinase with immunoglobulin-like and epidermal growth factor homology domains (TIE-2), insulin growth factor-I (IGFI) receptor, macrophage colony stimulating factor Cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath J. C., Exp. Opin. Ther. Patents, 10(6):803-818 (2000); Shawver L. K., et al., Drug Discov. Today, 2(2): 50-63 (1997); and Lofts, F. J. and Gullick W. J., “Growth factor receptors as targets.” in New Molecular Targets for Cancer Chemotherapy, Kerr D. J. and Workman P. (editors), (Jun. 27, 1994), CRC Press. Non-limiting examples of growth factor receptor inhibitors include pazopanib and sorafenib.

Pazopanib, 5-[[4-[(2,3-dimethyl-2H-indazol-6-yl)methylamino]-2-pyrimidinyl]amino]-2-methylbenzenesulfonamide, is a VEGFR inhibitor and is commercially available as VOTRIENT® tablets. Pazopanib was disclosed and claimed in International Application No. PCT/US01/49367, having an International filing date of Dec. 19, 2001, International Publication Number WO02/059110 and an International Publication date of Aug. 1, 2002, the entire disclosure of which is hereby incorporated by reference. Pazopanib is indicated for the treatment of advanced renal cell carcinoma and advanced soft tissue sarcoma. Grade 3 fatigue and hypertension are the most common dose limiting side effects of pazopanib.

Sorafenib, 4-[4-[[4-chloro-3-(trifluoromethyl)phenyl]carbamoylamino] phenoxy]-N-methyl-pyridine-2-carboxamide, is a multikinase inhibitor, and is commercially available as NEXAVAR® tablets. Sorafenib is indicated for the treatment of renal cell carcinoma, hepatocellular carcinoma, and certain differentiated thyroid carcinomas.

Tyrosine kinases, which are not growth factor receptor kinases, are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinha S. and Corey S. J., J. Hematother. Stem Cell Res., 8(5): 465-480 (2004) and Bolen, J. B., Brugge, J. S., Annu. Rev. Immunol., 15: 371-404 (1997).

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall T. E., J. Pharmacol. Toxicol. Methods, 34(3): 125-32 (1995).

Inhibitors of serine/threonine kinases include, but are not limited to, MAP kinase cascade blockers which include blockers of Raf kinases (rafk), Mitogen or Extracellular Regulated Kinase (MEKs), and Extracellular Regulated Kinases (ERKs); Protein kinase C family member blockers including blockers of PKCs (alpha, beta, gamma, epsilon, mu, lambda, iota, zeta); IkB kinases (IKKa, IKKb); PKB family kinases; AKT kinase family members; TGF beta receptor kinases; and mammaliam target of rapamycin (mTOR) inhibitors, including, but not limited to rapamycin (FK506) and rapalogs, RAD001 or everolimus (Afinitor), CCI-779 or temsirolimus, AP23573, AZD8055, WYE-354, WYE-600, WYE-687 and Pp121. Examples of inhibitors of serine/threonine kinases include, but are not limited to, trametinib, dabrafenib, and Akt inhibitors afuresertib and N-{(1S)-2-amino-1-[(3,4-difluorophenyl)methyl]ethyl}-5-chloro-4-(4-chloro-1-methyl-1H-pyrazol-5-yl)-2-furancarboxamide.

Trametinib, N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, is a MEK inhibitor and is commercially available as MEKINIST® tablets. Trametinib was disclosed and claimed in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference. Trametinib is indicated for the treatment of some unresectable or metastatic melanomas.

Dabrafenib, N-{3-[5-(2-Amino-4-pyrimidinyl)-2-(1,1-dimethylethyl)-1,3-thiazol-4-yl]-2-fluorophenyl}-2,6-difluorobenzenesulfonamide, is a B-Raf inhibitor and is commercially available as TAFINLAR® capsules. Dabrafenib was disclosed and claimed, in International Application No. PCT/US2009/042682, having an International filing date of May 4, 2009, the entire disclosure of which is hereby incorporated by reference. Dabrafenib is indicated for the treatment of some unresectable or metastatic melanomas.

Afuresertib, N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof, is an Akt inhibitor, and was disclosed and claimed in International Application No. PCT/US2008/053269, having an International filing date of Feb. 7, 2008; International Publication Number WO 2008/098104 and an International Publication date of Aug. 14, 2008, the entire disclosure of which is hereby incorporated by reference. Afuresertib can be prepared as described in International Application No. PCT/US2008/053269.

N-(1S)-2-amino-1-[(3,4-difluorophenyl)methyl]ethyl-5-chloro-4-(4-chloro-1-methyl-1H-pyrazol-5-yl)-2-furancarboxamide or a pharmaceutically acceptable salt thereof, is an Akt inhibitor, and was disclosed and claimed in International Application No. PCT/US2008/053269, having an International filing date of Feb. 7, 2008; International Publication Number WO 2008/098104 and an International Publication date of Aug. 14, 2008, the entire disclosure of which is hereby incorporated by reference. N-{(1S)-2-amino-1-[(3,4-difluorophenyl)methyl]ethyl}-5-chloro-4-(4-chloro-1-methyl-1H-pyrazol-5-yl)-2-furancarboxamide can be prepared as described in International Application No. PCT/US2008/053269.

Inhibitors of phosphatidyl inositol 3-kinase family members including blockers of PI3-kinase, ATM, DNA-PK, and Ku are also useful in the present invention. Such kinases are discussed in Abraham R. T., Curr. Opin. Immunol., 8(3): 412-418 (1996); Canman C. E., and Lim D. S., Oncogene, 17(25): 3301-3308 (1998); Jackson S. P., Int. J. Biochem. Cell Biol., 29(7): 935-938 (1997); and Zhong H., et al., Cancer Res., 60(6): 1541-1545 (2000).

Also useful in the present invention are myo-inositol signalling inhibitors such as phospholipase C blockers and myo-inositol analogs. Such signal inhibitors are described in Powis G., and Kozikowski A., “Inhibitors of Myo-Inositol Signaling.” in New Molecular Targets for Cancer Chemotherapy, Kerr D. J. and Workman P. (editors), (Jun. 27, 1994), CRC Press.

Another group of signal transduction pathway inhibitors are inhibitors of Ras oncogene. Such inhibitors include inhibitors of famesyltransferase, geranyl-geranyl transferase, and CAAX proteases as well as anti-sense oligonucleotides, ribozymes and other immunotherapies. Such inhibitors have been shown to block ras activation in cells containing wild type mutant ras, thereby acting as antiproliferation agents. Ras oncogene inhibition is discussed in Scharovsky O. G., et al., J. Biomed. Sci., 7(4): 292-298 (2000); Ashby M. N., Curr. Opin. Lipidol., 9(2): 99-102 (1998); and Bennett C. F. and Cowsert L. M., Biochim. Biophys. Acta., 1489(1): 19-30 (1999).

Antagonists to receptor kinase ligand binding may also serve as signal transduction inhibitors. This group of signal transduction pathway inhibitors includes the use of humanized antibodies or other antagonists to the extracellular ligand binding domain of receptor tyrosine kinases. Examples of antibody or other antagonists to receptor kinase ligand binding include, but are not limited to, cetuximab (ERBITUX®); trastuzumab (HERCEPTIN®); trastuzumab emtansine (KADCYLA®); pertuzumab (PERJETA®); ErbB inhibitors including lapatinib, erlotinib, and gefitinib; and 2C3 VEGFR2 specific antibody (see Brekken R. A., et al., Cancer Res., 60(18): 5117-5124 (2000)).

Cetuximab is a chimeric mouse human antibody which is commercially available as ERBITUX®. Cetuximab inhibits epidermal growth factor receptor (EGFR). Ceteximab in combination with radiation therapy is indicated for the treatment of squamous cell carcinoma of the head and neck, and is also indicated for the treatment of some colorectal cancers.

Trastuzumab is a humanized monoclonal antibody which is commercially available as HERCEPTIN®. Trastuzumab binds to the HER2 (also known as ErbB2) receptor. The original indication for trastuzumab is HER2 positive breast cancer.

Trastuzumab emtansine is an antibody-drug conjugate consisting of the monoclonal antibody trastuzumab (Herceptin®) linked to the cytotoxic agent emtansine (DM1), and is commercially available as an injectable solution KADCYLA®. Trastuzumab emtansine is indicated for the treatment of some HER2-positive metastatic brease breast cancers.

Pertuzumab is a monoclonal antibody which is commercially available as PERJETA®. Pertuzumab is a HER dimerization inhibitor, binding to HER2 to inhibit it from dimerizing with other HER receptors, which is hypothesized to result in slowed tumor growth. Pertuzumab is indicated in combination with trastuzumab (Herceptin) and docetaxel (TAXOTERE®) for the treatment of some HER2-positive metastatic breast cancers.

Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine is a dual inhibitor of ErbB-1 and ErbB-2 (EGFR and HER2) tyrosine kinases, and is commercially available as TYKERB® tablets. Lapatinib is indicated in combination with capecitabine (XELODA®) for the treatment of HER2-positive metastatic breast cancer.

Erlotinib, N-(3-ethynylphenyl)-6,7-bis{[2-(methyloxy)ethyl]oxy}-4-quinazolinamine, is an ErbB inhibitor, and is commercially available as TARCEVA® tablets. Erlotinib is indicated for the treatment of some locally advanced or metastatic non-small cell lung cancers, and for the treatment of some locally advanced, unresectable or metastatic pancreatic cancers, in combination with gemcitabine.

Gefitinib, N-(3-chloro-4-fluoro-phenyl)-7-methoxy-6-(3-morpholin-4-ylpropoxy)quinazolin-4-amine, is an ErbB-1 inhibitor, and is commercially available as IRESSA® tablets. Gefitinib is indicated as monotherapy for the treatment of patients with locally advanced or metastatic non-small-cell lung cancer after failure of both platinum-based and docetaxel chemotherapies.

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphav beta3) that will inhibit angiogenesis; endostatin and angiostatin (non-RTK) may also prove useful in combination with the disclosed compounds. (See Bruns C. J., et al., Cancer Res., 60(11): 2926-2935 (2000); Schreiber A. B., et al., Science, 232(4755): 1250-1253 (1986); Yen L., et al., Oncogene, 19(31): 3460-3469 (2000)).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R. T., et al., Cancer Res., 60(13): 3569-3576 (2000); and Chen Y., et al., Cancer Res., 58(9): 1965-1971 (1998).

Agents used in proapoptotic regimens (e.g., Bcl-2 antisense oligonucleotides) may also be used in the combination of the present invention. Members of the Bcl-2 family of proteins block apoptosis. Upregulation of Bcl-2 has therefore been linked to chemoresistance. Studies have shown that the epidermal growth factor (EGF) stimulates anti-apoptotic members of the Bcl-2 family (i.e., Mcl-1). Therefore, strategies designed to downregulate the expression of Bcl-2 in tumors have demonstrated clinical benefit. Such proapoptotic strategies using the antisense oligonucleotide strategy for Bcl-2 are discussed in Waters J. S., et al., J. Clin. Oncol., 18(9): 1812-1823 (2000); and Kitada S., et al., Antisense Res. Dev., 4(2): 71-79 (1994).

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania G. R., and Chang Y. T., Exp. Opin. Ther. Patents, 10(2): 215-230 (2000). Further, p21WAF1/CIP1 has been described as a potent and universal inhibitor of cyclin-dependent kinases (Cdks) (Ball K. L., Prog. Cell Cycle Res., 3: 125-134 (1997)). Compounds that are known to induce expression of p21WAF1/CIP1 have been implicated in the suppression of cell proliferation and as having tumor suppressing activity (Richon V. M., et al., Proc. Natl. Acad. Sci. USA, 97(18): 10014-10019 (2000)), and are included as cell cycle signaling inhibitors. Histone deacetylase (HDAC) inhibitors are implicated in the transcriptional activation of p21WAF1/CIP1 (Vigushin D. M., and Coombes R. C., Anticancer Drugs, 13(1): 1-13 (2002)), and are suitable cell cycle signaling inhibitors for use in combination herein. Examples of such HDAC inhibitors include, but are not limited to vorinostat, romidepsin, panobinostat, valproic acid, and mocetinostat.

Vorinostat, N-hydroxy-N′-phenyl-octanediamide, is a HDAC inhibitor, and is commercially available as ZOLINZA® capsules. Vorinostat is indicated for the treatment of cutaneous T-cell lymphoma (CTCL).

Romidepsin, (1S,4S,7Z,10S,16E,21R)-7-ethylidene-4,21-di(propan-2-yl)-2-oxa-12,13-dithia-5,8,20,23-tetrazabicyclo[8.7.6]tricos-16-ene-3,6,9,19,22-pentone, is a HDAC inhibitor, and is commercially available as an injectable solution as ISTODAX®. Romidepsin is indicated for the treatment of CTCL.

Panobinostat, (2E)-N-hydroxy-3-[4-({[2-(2-methyl-1H-indol-3-yl)ethyl]amino}methyl)phenyl]acrylamide, is a non-selective HDAC inhibitor, and is commercially available as FARYDAK® capsules. Panobinostat, in combination with bortezomib and dexamethasone, is indicated for the treatment of multiple myeloma.

Valproic acid, 2-propylpentanoic acid, is a HDAC inhibitor, and is commercially available as DEPAKENE® capsules, among others. Valproic acid is indicated as monotherapy and adjunctive therapy for the treatment of some seizures and has been explored for the treatment of various cancers.

Mocetinostat, N-(2-Aminophenyl)-4-[[(4-pyridin-3-ylpyrimidin-2-yl)amino]methyl]benzamide, is a benzamide HDAC inhibitor. Mecetinostat is currently undergoing clinical trials for the treatment of various cancers.

Proteasome inhibitors are drugs that block the action of proteasomes, cellular complexes that break down proteins, like the p53 protein. Several proteasome inhibitors are marketed or are being studied for the treatment of cancer. Suitable proteasome inhibitors for use in combination herein include, but are not limited to bortezomib, disulfiram, epigallocatechin gallate, salinosporamide A, and carfilzomib.

Bortezomib, [(1R)-3-methyl-1-({(2S)-3-phenyl-2-[(pyrazin-2-ylcarbonyl)amino]propanoyl}amino)butyl]boronic acid, is a proteasome inhibitor, and is commercially available as an injectable solution as VELCADE®. Bortezomib is indicated for the treatment of multiple myeloma and mantle cell lymphoma.

Disulfiram, 1,1′,1″,1′″-[disulfanediylbis(carbonothioylnitrilo)]tetraethane, is commercially available as ANTABUSE® tablets. Disulfiram is indicated as an aid in the management of sobriety in selected chronic alcohol patients. When disulfiram is complexed with metals to form dithiocarbamate complexes, it is a proteasome inhibitor, and such dithiocarbamate complexes have been explored for the treatment of various cancers (Cheriyan V. T., et al., PLoS One, 9(4): e93711 (2014)).

Epigallocatechin gallate (EGCG), [(2R,3R)-5,7-dihydroxy-2-(3,4,5-trihydroxyphenyl)chroman-3-yl]3,4,5-trihydroxybenzoate, is the most abundant catechin in tea, and is a proteasome inhibitor. EGCG has been explored for the treatment of various cancers (Yang H., et al., Curr. Cancer Drug Targets, 11(3): 296-306 (2011)).

Salinosporamide A, (4R,5S)-4-(2-chloroethyl)-1-((1 S)-cyclohex-2-enyl(hydroxy)methyl)-5-methyl-6-oxa-2-azabicyclo[3.2.0]heptane-3,7-dione, also known as marizomib, is a proteasome inhibitor. Salinosporamide A has been explored for the treatment of various cancers.

Carfilzomib, (2S)-4-Methyl-N-[(2S)-1-[[(2S)-4-methyl-1-[(2R)-2-methyloxiran-2-yl]-1-oxopentan-2-yl]amino]-1-oxo-3-phenylpropan-2-yl]-2-[[(2S)-2-[(2-morpholin-4-ylacetyl)amino]-4-phenylbutanoyl]amino]pentanamide, is a selective proteasome inhibitor, and is commercially available as an injectable solution as KYPROLIS®. Carfilzomib is indicated for the treatment of certain multiple myelomas.

The 70 kilodalton heat shock proteins (Hsp70s) and 90 kilodalton heat shock proteins (Hsp90s) are a family of ubiquitously expressed heat shock proteins. Hsp70s and Hsp90s are over expressed certain cancer types. Several Hsp70 and Hsp90 inhibitors are being studied in the treatment of cancer. Examples of Hsp70 and Hsp90 inhibitors for use in combination herein include, but are not limited to tanespimycin and radicicol.

Tanespimycin, 17-N-allylamino-17-demethoxygeldanamycin, is a derivative of the antibiotic geldanamycin, and is a Hsp90 inhibitor. Tanespimyicn has been explored for the treatment of various cancers.

Radicicol, [1aS-(1aR*,2Z,4E,14*, 15aR*)]-8-Chloro-1a,14,15,15a-tetrahydro-9,11-dihydroxy-14-methyl-6H-oxireno[e][2]benzoxacyclotetradecin-6,12(7H)-dione, also known as monorden, is a Hsp90 inhibitor. Radicicol has been explored for the treatment of various cancers.

Many tumor cells show a markedly different metabolism from that of normal tissues. For example, the rate of glycolysis, the metabolic process that converts glucose to pyruvate, is increased, and the pyruvate generated is reduced to lactate, rather than being further oxidized in the mitochondria via the tricarboxylic acid (TCA) cycle. This effect is often seen even under aerobic conditions and is known as the Warburg Effect.

Lactate dehydrogenase A (LDH-A), an isoform of lactate dehydrogenase expressed in muscle cells, plays a pivotal role in tumor cell metabolism by performing the reduction of pyruvate to lactate, which can then be exported out of the cell. The enzyme has been shown to be upregulated in many tumor types. The alteration of glucose metabolism described in the Warburg effect is critical for growth and proliferation of cancer cells and knocking down LDH-A using RNA-i has been shown to lead to a reduction in cell proliferation and tumor growth in xenograft models (Tennant D. A., et al., Nat. Rev. Cancer, 10(4): 267-277 (2010); Fantin V. R., et al., Cancer Cell, 9(6): 425-434 (2006)).

High levels of fatty acid synthase (FAS) have been found in cancer precursor lesions. Pharmacological inhibition of FAS affects the expression of key oncogenes involved in both cancer development and maintenance. Alli P. M., et al., Oncogene, 24(1): 39-46 (2005).

Inhibitors of cancer metabolism, including inhibitors of LDH-A and inhibitors of fatty acid biosynthesis (or FAS inhibitors), are suitable for use in combination herein.

Cancer gene therapy involves the selective transfer of recombinant DNA/RNA using viral or nonviral gene delivery vectors to modify cancer calls for therapeutic purposes. Examples of cancer gene therapy include, but are not limited to suicide and oncolytic gene therapies, as well as adoptive T-cell therapies.

Additional examples of a further active ingredient or ingredients (anti-neoplastic agent) for use in combination or co-administered with the present methods or combinations are antibodies or other antagonists to CD20, retinoids, or other kinase inhibitors. Examples of such antibodies or antagonists include, but are not limited to rituximab (RITUXAN® and MABTHERA®), ofatumumab (ARZERRA®), and bexarotene (TARGRETIN®).

Rituximab is a chimeric monoclonal antibody which is commercially available as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is commercially available as ARZERRA®. Ofatumumab binds to CD20 on B cells and is used to treat chronic lymphocytic leukemia CLL; a type of cancer of the white blood cells) in adults who are refractory to treatment with fludarabine (FLUDARA®) and alemtuzumab (CAMPATH®).

Bexarotene, 4-[1-(5,6,7,8-tetrahydro-3,5,5,8,8-pentamethyl-2-naphthalenyl)ethenyl]benzoic acid, is commercially available as TARGRETIN® capsules. Bexarotene is a member of a subclass of retinoids that selectively activate retinoid X receptors (RXRs). These retinoid receptors have biologic activity distinct from that of retinoic acid receptors (RARs). Bexarotene is indicated for the treatment of certain CTCLs.

Additional examples of a further active ingredient or ingredients (anti-neoplastic agent) for use in combination or co-administered with the present methods or combinations are Toll-like Receptor 4 (TLR4) antagonists.

Aminoalkyl glucosaminide phosphates (AGPs) are known to be useful as vaccine adjuvants and immunostimulatory agents for stimulating cytokine production, activating macrophages, promoting innate immune response, and augmenting antibody production in immunized animals. Aminoalkyl glucosaminide phosphates (AGPs) are synthetic ligands of the Toll-like Receptor 4 (TLR4). AGPs and their immunomodulating effects via TLR4 are disclosed in patent publications such as WO 2006016997, WO 2001090129, and/or U.S. Pat. No. 6,113,918 and have been reported in the literature. Additional AGP derivatives are disclosed in U.S. Pat. Nos. 7,129,219, 6,911,434, and 6,525,028. Certain AGPs act as agonists of TLR4, while others are recognized as TLR4 antagonists.

Select anti-neoplastic agents that may be used in combination with the present methods or combinations, include but are not limited to: abarelix, abemaciclib, abiraterone, afatinib, aflibercept, aldoxorubicin, alectinib, alemtuzumab, arsenic trioxide, asparaginase, axitinib, AZD-9291, belinostat, bendamustine, bevacizumab, blinatumomab, bosutinib, brentuximab vedotin, cabazitaxel, cabozantinib, capecitabine, ceritinib, clofarabine, cobimetinib, crizotinib, daratumumab, dasatinib, degarelix, denosumab, dinutuximab, docetaxel, elotuzumab, entinostat, enzalutamide, epirubicin, eribulin, filgrastim, flumatinib, fulvestrant, fruquintinib, gemtuzumab ozogamicin, ibritumomab, ibrutinib, idelalisib, imatinib, irinotecan, ixabepilone, ixazomib, lenalidomide, lenvatinib, leucovorin, mechlorethamine, necitumumab, nelarabine, netupitant, nilotinib, obinutuzumab, olaparib, omacetaxine, osimertinib, oxaliplatin, paclitaxel, palbociclib, palonosetron, panitumumab, pegfilgrastim, peginterferon alfa-2b, pemetrexed, plerixafor, pomalidomide, ponatinib, pralatrexate, quizartinib, radium-223, ramucirumab, regorafenib, rolapitant, rucaparib, sipuleucel-T, sonidegib, sunitinib, talimogene laherparepvec, tipiracil, topotecan, trabectedin, trifluridine, triptorelin, uridine, vandetanib, velaparib, vemurafenib, venetoclax, vincristine, vismodegib, and zoledronic acid.

### EXAMPLES

The following examples illustrate various non-limiting aspects of this invention.

### Example 1

### Arginine Methylation and PRMTs

Arginine methylation is an important post-translational modification on proteins involved in a diverse range of cellular processes such as gene regulation, RNA processing, DNA damage response, and signal transduction. Proteins containing methylated arginines are present in both nuclear and cytosolic fractions suggesting that the enzymes that catalyze the transfer of methyl groups on to arginines are also present throughout these subcellular compartments (reviewed in Yang, Y. & Bedford, M. T. Protein arginine methyltransferases and cancer. Nat Rev Cancer 13, 37-50, doi:10.1038/nrc3409 (2013); Lee, Y. H. & Stallcup, M. R. Minireview: protein arginine methylation of nonhistone proteins in transcriptional regulation. Mol Endocrinol 23, 425-433, doi:10.12 10/me.2008-0380 (2009)). In mammalian cells, methylated arginine exists in three major forms: ω-N^{G}-monomethyl-arginine (MMA), ω-N^{G},N^{G}-asymmetric dimethyl arginine (ADMA), or ω-N^{G},N′^{G}-symmetric dimethyl arginine (SDMA). Each methylation state can affect protein-protein interactions in different ways and therefore has the potential to confer distinct functional consequences for the biological activity of the substrate (Yang, Y. & Bedford, M. T. Protein arginine methyltransferases and cancer. Nat Rev Cancer 13, 37-50, doi:10.1038/nrc3409 (2013)).

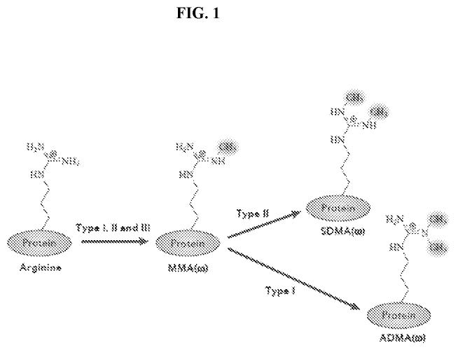

Arginine methylation occurs largely in the context of glycine-, arginine-rich (GAR) motifs through the activity of a family of Protein Arginine Methyltransferases (PRMTs) that transfer the methyl group from S-adenosyl-L-methionine (SAM) to the substrate arginine side chain producing S-adenosyl-homocysteine (SAH) and methylated arginine (FIG. 1). This family of proteins is comprised of 10 members of which 9 have been shown to have enzymatic activity (Bedford, M. T. & Clarke, S. G. Protein arginine methylation in mammals: who, what, and why. Mol Cell 33, 1-13, doi:10.1016/j.molcel.2008.12.013 (2009)). The PRMT family is categorized into four sub-types (Type I-IV) depending on the product of the enzymatic reaction (FIG. 1). Type IV enzymes methylate the internal guanidino nitrogen and have only been described in yeast (Fisk, J. C. & Read, L. K. Protein arginine methylation in parasitic protozoa. Eukaryot Cell 10, 1013-1022, doi:10.1128/EC.05103-11 (2011)); types I-III enzymes generate monomethyl-arginine (MMA, Rme1) through a single methylation event. The MMA intermediate is considered a relatively low abundance intermediate, however, select substrates of the primarily Type III activity of PRMT7 can remain monomethylated, while Types I and II enzymes catalyze progression from MMA to either asymmetric dimethyl-arginine (ADMA, Rme2a) or symmetric dimethyl arginine (SDMA, Rme2s) respectively. Type II PRMTs include PRMT5, and PRMT9, however, PRMT5 is the primary enzyme responsible for formation of symmetric dimethylation. Type I enzymes include PRMT1, PRMT3, PRMT4, PRMT6 and PRMT8. PRMT1, PRMT3, PRMT4, and PRMT6 are ubiquitously expressed while PRMT8 is largely restricted to the brain (reviewed in Bedford, M. T. & Clarke, S. G. Protein arginine methylation in mammals: who, what, and why. Mol Cell 33, 1-13, doi:10.1016/j.molcel.2008.12.013 (2009)).

PRMT1 is the primary Type 1 enzyme capable of catalyzing the formation of MMA and ADMA on numerous cellular substrates (Bedford, M. T. & Clarke, S. G. Protein arginine methylation in mammals: who, what, and why. Mol Cell 33, 1-13, doi:10.1016/j.molcel.2008.12.013 (2009)). In many instances, the PRMT1-dependent ADMA modification is required for the biological activity and trafficking of its substrates (Nicholson, T. B., Chen, T. & Richard, S. The physiological and pathophysiological role of PRMT1-mediated protein arginine methylation. Pharmacol Res 60, 466-474, doi:10.1016/j.phrs.2009.07.006 (2009)), and the activity of PRMT1 accounts for ˜85% of cellular ADMA levels (Dhar, S. et al. Loss of the major Type I arginine methyltransferase PRMT1 causes substrate scavenging by other PRMTs. Sci Rep 3, 1311, doi:10.1038/srep01311 (2013); Pawlak, M. R., Scherer, C. A., Chen, J., Roshon, M. J. & Ruley, H. E. Arginine N-methyltransferase 1 is required for early postimplantation mouse development, but cells deficient in the enzyme are viable. Mol Cell Biol 20, 4859-4869 (2000)). Complete knockout of PRMT1 results in a profound increase in MMA across numerous substrates suggesting that the major biological function for PRMT1 is to convert MMA to ADMA while other PRMTs can establish and maintain MMA (Dhar, S. et al. Loss of the major Type I arginine methyltransferase PRMT1 causes substrate scavenging by other PRMTs. Sci Rep 3, 1311, doi:10.1038/srep01311 (2013)). In addition, SDMA levels are increased upon loss of PRMT1, likely a consequence of the loss of ADMA and the corresponding increase of MMA that can serve as the substrate for SDMA-generating Type II PRMTs. Inhibition of Type I PRMTs may lead to altered substrate function through loss of ADMA, increase in MMA, or, alternatively, a switch to the distinct methylation pattern associated with SDMA (Dhar, S. et al. Loss of the major Type I arginine methyltransferase PRMT1 causes substrate scavenging by other PRMTs. Sci Rep 3, 1311, doi:10.1038/srep01311 (2013)).

Disruption of the Prmt1 locus in mice results in early embryonic lethality and homozygous embryos fail to develop beyond E6.5 indicating a requirement for PRMT1 in normal development (Pawlak, M. R., Scherer, C. A., Chen, J., Roshon, M. J. & Ruley, H. E. Arginine N-methyltransferase 1 is required for early postimplantation mouse development, but cells deficient in the enzyme are viable. Mol Cell Biol 20, 4859-4869 (2000); Yu, Z., Chen, T., Hebert, J., Li, E. & Richard, S. A mouse PRMT1 null allele defines an essential role for arginine methylation in genome maintenance and cell proliferation. Mol Cell Biol 29, 2982-2996, doi:10.1128/MCB.00042-09 (2009)). Conditional or tissue specific knockout will be required to better understand the role for PRMT1 in the adult. Mouse embryonic fibroblasts derived from Prmt1 null mice undergo growth arrest, polyploidy, chromosomal instability, and spontaneous DNA damage in association with hypomethylation of the DNA damage response protein MRE11, suggesting a role for PRMT1 in genome maintenance and cell proliferation (Yu, Z., Chen, T., Hebert, J., Li, E. & Richard, S. A mouse PRMT1 null allele defines an essential role for arginine methylation in genome maintenance and cell proliferation. Mol Cell Biol 29, 2982-2996, doi:10.1128/MCB.00042-09 (2009)). PRMT1 protein and mRNA can be detected in a wide range of embryonic and adult tissues, consistent with its function as the enzyme responsible for the majority of cellular arginine methylation. Although PRMTs can undergo post-translational modifications themselves and are associated with interacting regulatory proteins, PRMT1 retains basal activity without a requirement for additional modification (reviewed in Yang, Y. & Bedford, M. T. Protein arginine methyltransferases and cancer. Nat Rev Cancer 13, 37-50, doi:10.1038/nrc3409 (2013)).

### PRMT1 and Cancer

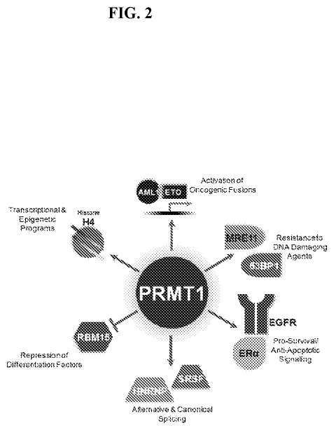

Mis-regulation and overexpression of PRMT1 has been associated with a number of solid and hematopoietic cancers (Yang, Y. & Bedford, M. T. Protein arginine methyltransferases and cancer. Nat Rev Cancer 13, 37-50, doi:10.1038/nrc3409 (2013); Yoshimatsu, M. et al. Dysregulation of PRMT1 and PRMT6, Type I arginine methyltransferases, is involved in various types of human cancers. Int J Cancer 128, 562-573, doi:10.1002/ijc.25366 (2011)). The link between PRMT1 and cancer biology has largely been through regulation of methylation of arginine residues found on relevant substrates (FIG. 2). In several tumor types, PRMT1 can drive expression of aberrant oncogenic programs through methylation of histone H4 (Takai, H. et al. 5-Hydroxymethylcytosine plays a critical role in glioblastomagenesis by recruiting the CHTOP-methylosome complex. Cell Rep 9, 48-60, doi:10.1016/j.celrep.2014.08.071 (2014); Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Zhao, X. et al. Methylation of RUNX1 by PRMT1 abrogates SIN3A binding and potentiates its transcriptional activity. Genes Dev 22, 640-653, doi:10.1101/gad.1632608 (2008)), as well as through its activity on non-histone substrates (Wei, H., Mundade, R., Lange, K. C. & Lu, T. Protein arginine methylation of non-histone proteins and its role in diseases. Cell Cycle 13, 32-41, doi:10.4161/cc.27353 (2014)). In many of these experimental systems, disruption of the PRMT1-dependent ADMA modification of its substrates decreases the proliferative capacity of cancer cells (Yang, Y. & Bedford, M. T. Protein arginine methyltransferases and cancer. Nat Rev Cancer 13, 37-50, doi:10.1038/nrc3409 (2013)).

Several studies have linked PRMT1 to the development of hematological and solid tumors. PRMT1 is associated with leukemia development through methylation of key drivers such as MLL and AML1-ETO fusions, leading to activation of oncogenic pathways (Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Cheung, N. et al. Targeting Aberrant Epigenetic Networks Mediated by PRMT1 and KDM4C in Acute Myeloid Leukemia. Cancer Cell 29, 32-48, doi:10.1016/j.ccell.2015.12.007 (2016)). Knockdown of PRMT1 in bone marrow cells derived from AML-ETO expressing mice suppressed clonogenicity, demonstrating a critical requirement for PRMT1 in maintaining the leukemic phenotype of this model (Shia, W. J. et al. PRMT1 interacts with AML-ETO to promote its transcriptional activation and progenitor cell proliferative potential. Blood 119, 4953-4962, doi:10.1182/blood-2011-04-347476 (2012)). PRMT1 is also a component of MLL fusion complexes, promotes aberrant transcriptional activation in association with H4R3 methylation, and knockdown of PRMT1 can suppress MLL-EEN mediated transformation of hematopoietic stem cells (Cheung, N., Chan, L. C., Thompson, A., Cleary, M. L. & So, C. W. Protein arginine-methyltransferase-dependent oncogenesis. Nat Cell Biol 9, 1208-1215, doi:10.1038/ncb1642 (2007)). In breast cancer patients, high expression of PRMT1 was found to correlate with shorter disease free survival and with tumors of advanced histological grade (Mathioudaki, K. et al. Clinical evaluation of PRMT1 gene expression in breast cancer. Tumour Biol 32, 575-582, doi:10.1007/s13277-010-0153-2 (2011)). To this end, PRMT1 has been implicated in the promotion of metastasis and cancer cell invasion (Gao, Y. et al. The dual function of PRMT1 in modulating epithelial-mesenchymal transition and cellular senescence in breast cancer cells through regulation of ZEB1. Sci Rep 6, 19874, doi:10.1038/srep19874 (2016); Avasarala, S. et al. PRMT1 Is a Novel Regulator of Epithelial-Mesenchymal-Transition in Non-small Cell Lung Cancer. J Biol Chem 290, 13479-13489, doi:10.1074/jbc.M114.636050 (2015)) and PRMT1 mediated methylation of Estrogen Receptor α (ERα) can potentiate growth-promoting signal transduction pathways. This methylation driven mechanism may provide a growth advantage to breast cancer cells even in the presence of anti-estrogens (Le Romancer, M. et al. Regulation of estrogen rapid signaling through arginine methylation by PRMT1. Mol Cell 31, 212-221, doi:10.1016/j.molcel.2008.05.025 (2008)). In addition, PRMT1 promotes genome stability and resistance to DNA damaging agents through regulating both homologous recombination and non-homologous end-joining DNA repair pathways (Boisvert, F. M., Rhie, A., Richard, S. & Doherty, A. J. The GAR motif of 53BP1 is arginine methylated by PRMT1 and is necessary for 53BP1 DNA binding activity. Cell Cycle 4, 1834-1841, doi:10.4161/cc.4.12.2250 (2005); Boisvert, F. M., Dery, U., Masson, J. Y. & Richard, S. Arginine methylation of MRE11 by PRMT1 is required for DNA damage checkpoint control. Genes Dev 19, 671-676, doi:10.1101/gad.1279805 (2005)). Therefore, inhibition of PRMT1 may sensitize cancers to DNA damaging agents, particularly in tumors where DNA repair machinery may be compromised by mutations (such as BRCA1 in breast cancers) (O'Donovan, P. J. & Livingston, D. M. BRCA1 and BRCA2: breast/ovarian cancer susceptibility gene products and participants in DNA double-strand break repair. Carcinogenesis 31, 961-967, doi:10.1093/carcin/bgq069 (2010)). Together, these observations demonstrate key roles for PRMT1 in clinically-relevant aspects of tumor biology, and suggest a rationale for exploring combinations with therapies such as those that promote DNA damage.

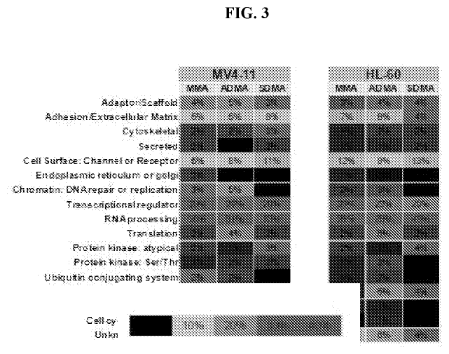

RNA binding proteins and splicing machinery are a major class of PRMT1 substrates and have been implicated in cancer biology through their biological function as well as recurrent mutations in leukemias (Bressan, G. C. et al. Arginine methylation analysis of the splicing-associated SR protein SFRS9/SRP30C. Cell Mol Biol Lett 14, 657-669, doi:10.2478/s11658-009-0024-2 (2009); Sveen, A., Kilpinen, S., Ruusulehto, A., Lothe, R. A. & Skotheim, R. I. Aberrant RNA splicing in cancer; expression changes and driver mutations of splicing factor genes. Oncogene 35, 2413-2427, doi:10.1038/onc.2015.318 (2016); Hsu, T. Y. et al. The spliceosome is a therapeutic vulnerability in MYC-driven cancer. Nature 525, 384-388, doi:10.1038/nature14985 (2015)). In a recent study, PRMT1 was shown to methylate the RNA binding protein, RBM15, in acute megakaryocytic leukemia (Zhang, L. et al. Cross-talk between PRMT1-mediated methylation and ubiquitylation on RBM15 controls RNA splicing. Elife 4, doi:10.7554/eLife.07938 (2015)). PRMT1 mediated methylation of RBM15 regulates its expression; consequently, overexpression of PRMT1 in AML cell lines was shown to block differentiation by downregulation of RBM15, thereby preventing its ability to bind pre-mRNA intronic regions of genes important for differentiation. To identify putative PRMT1 substrates, a proteomic approach (Methylscan, Cell Signaling Technology) was utilized to identify proteins with changes in arginine methylation states in response to a tool PRMT1 inhibitor, Compound D. Protein fragments from Compound D- and DSMO-treated cell extracts were immunoprecipitated using methyl arginine specific antibodies (ADMA, MMA, SDMA), and peptides were identified by mass spectrometry. While many proteins undergo changes in arginine methylation, the majority of substrates identified were transcriptional regulators and RNA processing proteins in AML cell lines treated with the tool compound (FIG. 3).

In summary, the impact of PRMT1 on cancer relevant pathways suggests inhibition may lead to anti-tumor activity, providing a novel therapeutic mechanism for the treatment of AML, lymphoma, and solid tumor indications. As described in the emerging literature, several mechanisms support a rationale for the use of a PRMT1 inhibitor in hematological and solid tumors including: inhibition of AML-ETO driven oncogenesis in leukemia, inhibition of growth promoting signal transduction in breast cancer, and modulation of splicing through methylation of RNA binding proteins and spliceosome machinery.

Inhibition of Type I PRMTs including PRMT1 represents a tractable strategy to suppress aberrant cancer cell proliferation and survival.

### Biochemistry

Detailed in vitro biochemical studies were conducted with Compound A to characterize the potency and mechanism of inhibition against Type I PRMTs.

### Mechanism of Inhibition

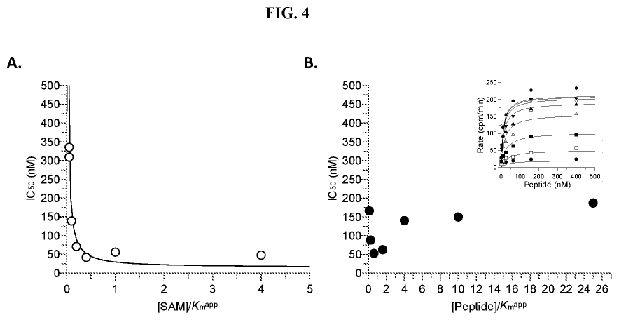

The inhibitory mechanism of Compound A for PRMT1 was explored through substrate competition experiments. Inhibitor modality was examined by plotting Compound A IC₅₀ values as a function of substrate concentration divided by its Kₘ^{app} and comparing the resulting plots to the Cheng-Prusoff relationship for competitive, non-competitive, and uncompetitive inhibition (Copeland, R. A. Evaluation of enzyme inhibitors in drug discovery. A guide for medicinal chemists and pharmacologists. Methods Biochem Anal 46, 1-265 (2005)). Compound A IC₅₀ values decreased with increasing SAM concentration indicating that inhibition of PRMT1 by Compound A was uncompetitive with respect to SAM with a Kᵢ^{app} value of 15 nM when fit to an equation for uncompetitive inhibition (FIG. 4A). No clear modality trend was observed when Compound A IC₅₀ values were plotted as a function of H4 1-21 peptide (FIG. 4B) suggesting mixed type inhibition. Further analysis was performed using a global analysis resulting in an a value of 3.7 confirming the peptide mechanism as mixed and yielding a Kᵢ^{app} value of 19 nM (FIG. 4B, inset).

### Time Dependence and Reversibility

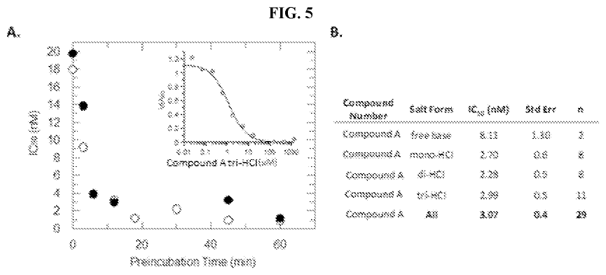

Compound A was evaluated for time dependent inhibition by measuring IC₅₀ values following varying SAM:PRMT1:Compound A preincubation time and a 20 minute reaction. An inhibitory mechanism that is uncompetitive with SAM implies that generation of the SAM:PRMT1 complex is required to support binding of Compound A, therefore SAM (held at Kₘ^{app}) was included during the preincubation. Compound A demonstrated time dependent inhibition of PRMT1 methylation evident by an increase in potency with longer preincubation time (FIG. 5A). Since time dependent inhibition was observed, further IC₅₀ determinations included a 60 minute SAM:PRMT1:Compound A preincubation and a 40 minute reaction time to provide a better representation of compound potency. These conditions yield an IC₅₀ of 3.1±0.4 nM (n=29) that is >10-fold above the theoretical tight-binding limit (0.25 nM) of the assay. Examining IC₅₀ values at varying PRMT1 concentrations revealed that the actual tight binding limit would be significantly lower than 0.25 nM potentially due to a low active fraction (FIG. 5B). The salt form of Compound A did not significantly affect the IC₅₀ value determined against PRMT1 (FIG. 5B).

Two explanations for time dependent inhibition are slow-binding reversible inhibition and irreversible inhibition. To distinguish between these two mechanisms, affinity selection mass spectrometry (ASMS) was used to examine the binding of Compound A to PRMT1. ASMS first separates bound from unbound ligand, and then detects reversibly bound ligand by MS. A 2 hr preincubation of PRMT1:SAM with Compound A was used to ensure that the time dependent complex (ESI*) was fully formed based on the profile shown in FIG. 5A) in which maximal potency was observed after 20 minutes of preincubation. Under these conditions, Compound A was detectable using ASMS. This suggests that the primary mechanism is reversible in nature, since ASMS would be unable to detect irreversibly bound Compound A. Definitive reversibility studies including off-rate analysis have not yet been performed and would further validate the mechanism.

### Crystallography

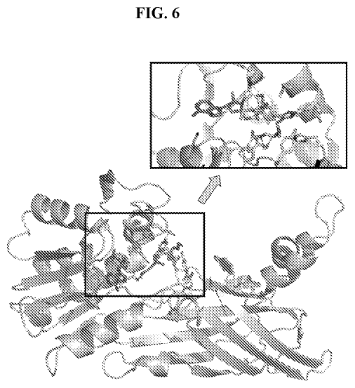

To determine inhibitor binding mode, the co-crystal structure of Compound A bound to PRMT1 and SAH was determined (2.48 Å resolution) (FIG. 6). SAH is the product formed upon removal of the methyl group from SAM by PRMT1; therefore, SAH and SAM should similarly occupy the same pocket of PRMT1. The inhibitor binds in the cleft normally occupied by the substrate peptide directly adjacent to the SAH pocket and its diamine sidechain occupies the putative arginine substrate site. The terminal methylamine forms a hydrogen bond with the Glu162 sidechain residue that is 3.6 Å from the thioether of SAH and the SAH binding pocket is bridged to Compound A by Tyr57 and Met66. Compound A binds PRMT1 through the formation of a hydrogen bond between the proton of the pyrazole nitrogen of Compound A and the acidic sidechain of Glu65; the diethoxy branched cyclohexyl moiety lies along the solvent exposed surface in a hydrophobic groove formed by Tyr57, Ile62, Tyr166 and Tyr170. The spatial separation between SAH and inhibitor binding, as well as interactions with residues such as Tyr57 could support the SAM uncompetitive mechanism revealed in the enzymatic studies. The finding that Compound A is bound in the substrate peptide pocket and that the diamine sidechain may mimic the amines of the substrate arginine residue implies that inhibitor modality may be competitive with peptide. Biochemical mode of inhibition studies support that Compound A is a mixed inhibitor with respect to peptide (FIG. 4B). The time-dependent behavior of Compound A as well as the potential for exosite binding of the substrate peptide outside of the peptide cleft could both result in a mode of inhibition that is not competitive with peptide, explaining the difference in modality suggested by the structural and biochemical studies.

### Orthologs

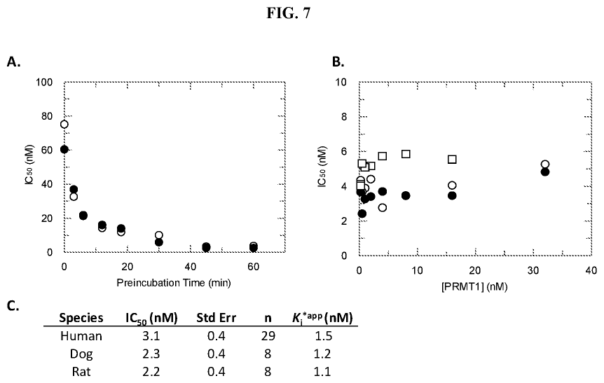

To facilitate interpretation of toxicology studies, the potency of Compound A was evaluated against the rat and dog orthologs of PRMT1. As with human PRMT1, Compound A revealed time dependent inhibition against rat and dog PRMT1 with IC₅₀ values decreasing with increasing preincubation (FIG. 7A). Additionally, no shift in Compound A potency was observed across a range of enzyme concentrations (0.25-32 nM) suggesting the IC₅₀ values measured did not approach the tight-binding limit of the assay for human, rat or dog (FIG. 7B). IC₅₀ values were determined using conditions equivalent to those used to assess human PRMT1 and revealed that Compound A potency varied <2-fold across all species (FIG. 7C).

### Selectivity

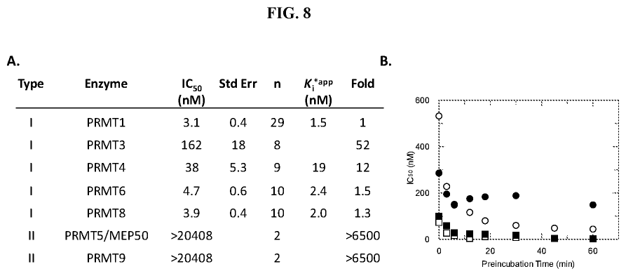

The selectivity of Compound A was assessed across a panel of PRMT family members. IC₅₀ values were determined against representative Types I (PRMT3, PRMT4, PRMT6 and PRMT8) and II (PRMT5/MEP50 and PRMT9) family members following a 60 minute SAM:Enzyme:Compound A preincubation. Compound A inhibited the activity of all Type I PRMTs tested with varying potencies, but failed to inhibit Type II family members (FIG. 8A). Additional characterization of the Type I PRMTs revealed that Compound A was a time dependent inhibitor of PRMT4, PRMT6 and PRMT8 due to the increase in potency observed following increasing Enzyme:SAM:Compound A preincubation times; whereas, PRMT3 displayed no time dependent behavior (FIG. 8B).

To further characterize selectivity of Compound A, the inhibition of twenty-one methyltransferases was evaluated at a single concentration of Compound A (10 μM, Reaction Biology). The highest degree of inhibition, 18%, was observed against PRDM9. Overall, Compound A showed minimal inhibition of the methyltransferases tested suggesting it is a selective inhibitor of Type I PRMTs (Table 1). Additional selectivity assays are described in the Safety sections.

TABLE 1
Methyltransferases tested for inhibition by Compound A.

| | | Average % |
|---|---|---|
| Methyltransferase | Substrate | Inhibition |
| PRDM9 | Histone H3 | 17.99 |
| NSD2 | Nucleosomes | 14.97 |
| MLL3 Complex | Core Histone | 13.67 |
| EZH1 Complex | Core Histone | 11.97 |
| SMYD2 | Histone H4 | 9.26 |
| PRMT3 | Histone H4 | 9.01 |
| EZH2 Complex | Core Histone | 8.17 |
| MLL2 Complex | Core Histone | 6.21 |
| SET1B Complex | Core Histone | 5.96 |
| NSD1 | Nucleosomes | 3.81 |
| G9a | Histone H3 (1-21) | 3.72 |
| SET7 | Core Histone | 3.47 |
| SETD2 | Nucleosomes | 3.15 |
| Dot1L | Nucleosomes | 2.75 |
| GLP | Histone H3 (1-21) | 1.86 |
| MLL4 Complex | Core Histone | 0.27 |
| MLL1 Complex | Nucleosomes | 0.27 |
| SUV420H1-tv2 | Nucleosomes | 0.00 |
| SUV39H1 | Histone H3 | 0.00 |
| SET8 | Nucleosomes | 0.00 |
| SUV39H2 | Histone H3 | 0.00 |

Enzymes were assayed at a fixed concentration of SAM (1 μM) independent of the SAM Km value.

In summary, Compound A is a potent, reversible, selective inhibitor of Type I PRMT family members showing equivalent biochemical potency against PRMT1, PRMT6 and PRMT8 with IC₅₀ values ranging between 3-5 nM. The crystal structure of PRMT1 in complex with Compound A reveals that Compound A binds in the peptide pocket and both the crystal structure, as well as enzymatic studies are consistent with a SAM uncompetitive mechanism.

### Biology

### Cellular Mechanistic Effects

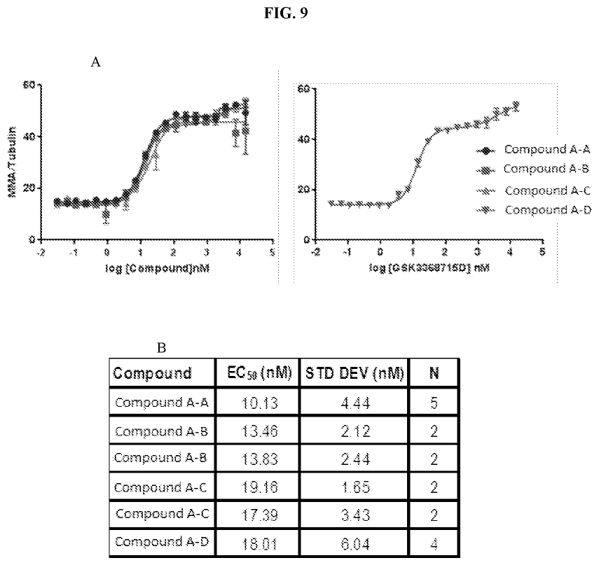

Inhibition of PRMT1 is predicted to result in a decrease of ADMA on cellular PRMT1 substrates, including arginine 3 of histone H4 (H4R3me2a), with concomitant increases in MMA and SDMA (Dhar, S. et al. Loss of the major Type I arginine methyltransferase PRMT1 causes substrate scavenging by other PRMTs. Sci Rep 3, 1311, doi:10.1038/srep01311 (2013)). To evaluate the effect of Compound A on arginine methylation the dose response associated with increased MMA was evaluated in an in-cell-western assay using an antibody to detect MMA and the cellular mechanistic EC₅₀ of 10.1±4.4 nM was determined (FIG. 9). The dose response appeared biphasic, possibly due to differential activity between the Type I PRMTs or differential potency towards a particular subset of substrates. An equation describing a biphasic curve was used to fit the data and since there was no obvious plateau associated with the second inflection over the range of concentrations tested, the first inflection was reported. Various salt forms were tested in this assay format and all demonstrated similar EC₅₀ values and are, therefore, considered interchangeable for all biology studies (FIG. 9). Additional studies were performed to examine the timing, durability, and impact on other methylation states in select tumor types as indicated below. The potency of Compound A on induction of MMA indicates that Compound A can be used to investigate the biological mechanism associated with inhibition of Type 1 PRMTs in cells.

### Type I PRMT Expression in Cancer

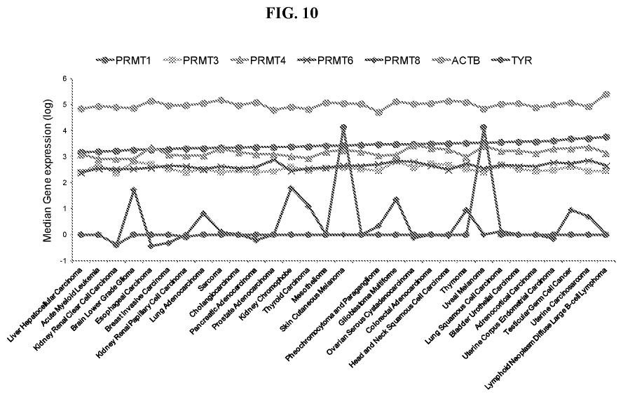

Analysis of gene expression data from multiple tumor types collected from >100 cancer studies through The Cancer Genome Atlas (TCGA) and other primary tumor databases represented in cBioPortal indicates that PRMT1 is highly expressed g in cancer, with highest levels in lymphoma (diffuse large B-cell lymphoma, DLBCL) relative to other solid and hematological malignancies (FIG. 10). Expression of ACTB, a common housekeeping gene and TYR, a gene selectively expressed in skin, were also surveyed to characterize the range associated with high ubiquitous expression or tissue restricted expression, respectively. High expression in lymphoma among other cancers provides additional confidence that the target of Compound A inhibition is present in primary tumors that correspond to cell lines evaluated in preclinical studies. PRMTs 3, 4, and 6 are also expressed across a range of tumor types while PRMT8 expression appears more restricted as predicted given its tissue specific expression (Lee, J., Sayegh, J., Daniel, J., Clarke, S. & Bedford, M. T. PRMT8, a new membrane-bound tissue-specific member of the protein arginine methyltransferase family. J Biol Chem 280, 32890-32896, doi:10.1074/jbc.M506944200 (2005)).

### Cellular Phenotypic Effects

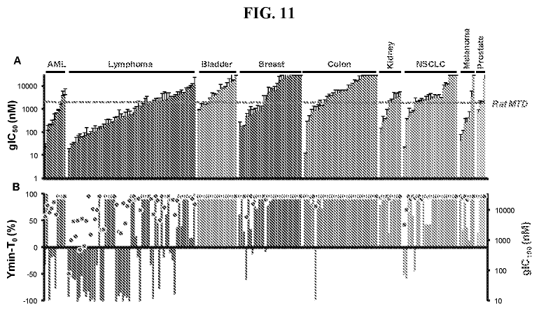

Compound A was analyzed for its ability to inhibit cultured tumor cell line growth in a 6-day growth-death assay using Cell Titer Glo (Promega) that quantifies ATP as a surrogate of cell number. The growth of all cell lines was evaluated over time across a wide range of seeding densities to identify conditions that permitted proliferation throughout the entire 6-day assay. Cells were plated at the optimal seeding density and after overnight incubation, a 20-point 2-fold titration of compound was added and plates were incubated for 6 days. A replicate plate of cells was harvested at the time of compound addition to quantify the starting number of cells (To). Values obtained after the 6 day treatment were expressed as a function of the To value and plotted against compound concentration. The To value was normalized to 100% and represents the number of cells at the time of compound addition. The data were fit with a 4 parameter equation to generate a concentration response curve and the growth IC₅₀ (gIC₅₀) was determined. The gIC₅₀ is the midpoint of the ‘growth window’, the difference between the number of cells at the time of compound addition (T₀) and the number of cells after 6 days (DMSO control). The growth-death assay can be used to quantify the net population change, clearly defining cell death (cytotoxicity) as fewer cells compared to the number at the time of compound addition (T₀). A negative Yₘᵢₙ−T₀ value is indicative of cell death while a gIC₁₀₀ value represents the concentration of compound required for 100% inhibition of growth. The growth inhibitory effect of Compound A was evaluated using this assay in 196 human cancer cell lines representing solid and hematological malignancies (FIG. 11).

Compound A induced near or complete growth inhibition in most cell lines, with a subset showing cytotoxic responses, as indicated by a negative Yₘᵢₙ−T₀ value (FIG. 11B). This effect was most pronounced in AML and lymphoma cancer cell lines, where 50 and 54% of cell lines showed cytotoxic responses, respectively. The total AUC or exposure (Cₐᵥₑ) calculated from the rat 14-day MTD (150 mg/kg, Cₐᵥₑ=2.1 μM) was used as an estimate of a clinically relevant concentration of Compound A for evaluation of sensitivity. While lymphoma cell lines showed cytotoxicity with gIC₁₀₀ values below 2.1 μM, many cell lines across all tumor types evaluated showed gIC₅₀ values ≤2.1 μM suggesting that concentrations associated with anti-tumor activity may be achievable in patients. The dog 21-day MTD was slightly higher (25 mg/kg; total AUC or Cₐᵥₑ=3.2 μM), therefore the lower concentration from the rat provides a more conservative target for appreciating cell line sensitivity. Lymphoma cell lines were highly sensitive to Type I PRMT inhibition, with a median gIC₅₀ of 0.57 μM and cytotoxicity observed in 54%. Among solid tumor types, potent anti-proliferative activity of Compound A was observed in melanoma and kidney cancer cell lines (primarily representing clear cell renal carcinoma), however, the responses were predominantly cytostatic in this assay format (FIG. 11, Table 2).

TABLE 2
Compound A 6-day proliferation summary.

| | Total | AML | Lymphoma | Bladder | Breast | Colon | Kidney | NSC-LC | Melanoma | Prostate |
|---|---|---|---|---|---|---|---|---|---|---|
| Median gIC₅₀ (μM) | 2.12 | 0.54 | 0.57 | 5.32 | 5.95 | 5.51 | 1.66 | 2.81 | 0.28 | 1.86 |
| Median gIC₁₀₀ (μM) | 29.33 | 16.72 | 21.62 | 29.33 | 29.36 | 29.33 | 29.35 | 29.33 | 29.33 | 29.34 |
| % Cytotoxic | 23% | 50% | 54% | 0% | 10% | 3% | 0% | 16% | 0% | 0% |
| % gIC₅₀ < 2 μM | 49% | 80% | 69% | 28% | 41% | 29% | 60% | 28% | 71% | 75% |
| % gIC₁₀₀ < 2 μM | 4% | 0% | 14% | 0% | 0% | 0% | 0% | 0% | 0% | 0% |
| Total Cell Lines | 196 | 10 | 59 | 18 | 29 | 34 | 10 | 25 | 7 | 4 |

gIC₅₀ ≤ 2.1 μM was used as target based on concentration achieved in the rat 14-day MTD (150 mg/kg, Cₐᵥₑ = 2.1 μM).

Evaluation of the anti-proliferative effects of Compound A indicates that inhibition S of PRMT1 results in potent anti-tumor activity across cell lines representing a range of solid and hematological malignancies. Together, these data suggest that clinical development in solid and hematological malignancies is warranted. Prioritized indications include:
Lymphoma: cytotoxicity in 54% of cell lines
AML: cytotoxicity in 50% of cell lines
Renal cell carcinoma: gIC₅₀≤2.1 μM in 60% of cell lines
Melanoma: gIC₅₀≤2.1 μM in 71% of cell lines
Breast cancer including TNBC: gIC₅₀≤2.1 μM in 41% of cell lines

### Lymphoma Biology

### Cell Mechanistic Effects

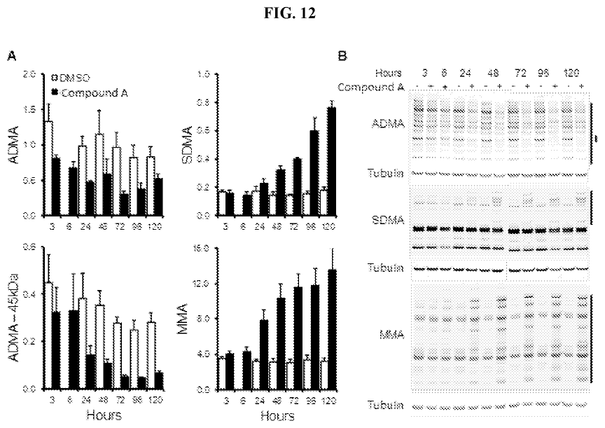

To evaluate the effect of Compound A on arginine methylation in lymphoma, a human DLBCL cell line (Toledo) was treated with 0.4 μM Compound A or vehicle for up to 120 hours after which protein lysates were evaluated by western analysis using antibodies for various arginine methylation states. As predicted, ADMA methylation decreased while MMA increased upon compound exposure (FIG. 12). An increase in levels of SDMA was also observed, suggesting that the increase in MMA may have resulted in accumulation in the pool of potential substrates for PRMT5, the major catalyst of SDMA formation. Given the detection of numerous substrates with varying kinetics, and variability of ADMA levels among DMSO-treated samples, both the full lane and a prominent 45 kDa band were characterized to assess ADMA. Increases in MMA were apparent by 24 hours and near maximal by 48 hours while decreases in the 45 kDa ADMA band required 72-96 hours to achieve maximal effect. Increases in SDMA were apparent after 48 hours of compound exposure and continued to increase through 120 hours, consistent with the potential switch from conversion of MMA to ADMA by Type I PRMTs to SDMA by Type II PRMTs (FIG. 12).

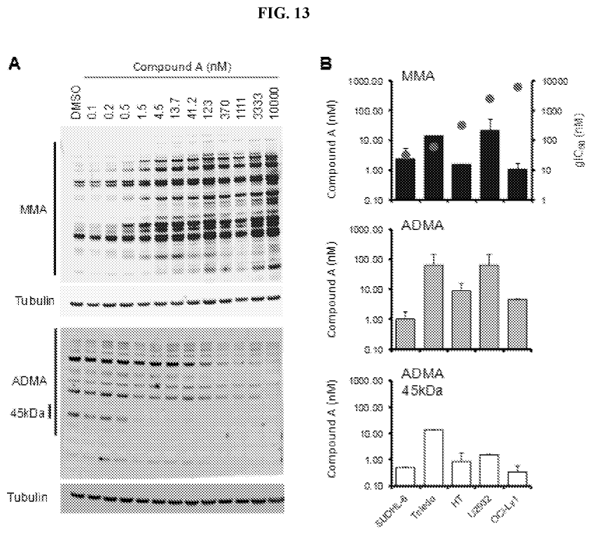

The dose response associated with Compound A effects on arginine methylation (MMA, ADMA, SDMA) was determined in a panel of lymphoma cell lines (FIG. 13). ADMA decreases were measured across the full lane and the single 45 kDa band that decreased to undetectable levels across all cell lines evaluated. Overall, concentrations required to achieve 50% of the maximal effect were similar across cell lines and did not correspond to the gIC₅₀ in the 6-day growth death assay, suggesting that the lack of sensitivity is not explained by poor target engagement.

To determine the durability of global changes in arginine methylation in response to Compound A, ADMA, SDMA, and MMA levels were assessed in cells treated with Compound A after compound washout (FIG. 14). Toledo cells were cultured with 0.4 μM Compound A for 72 hours to establish robust effects on arginine methylation marks. Cells were then washed, cultured in Compound A-free media, samples were collected daily through 120 hours, and arginine methylation levels were examined by western analysis. MMA levels rapidly decreased, returning to baseline by 24 hours after Compound A washout, while ADMA and SDMA returned to baseline by 24 and 96 hours, respectively. Notably, recovery of the 45 kDa ADMA band appeared delayed relative to most other species in the ADMA western blots, suggesting the durability of arginine methylation changes by Compound A may vary by substrate. SDMA appeared to continue to increase even after 6 hours of washout. This is consistent with the continued increase observed through 120 hours without any obvious plateau (FIG. 12) coupled with the durable increase in MMA that has not yet returned to baseline after washout. Durability of each modification generally reflected the kinetics of arginine methylation changes brought about by Compound A, with MMA being the most rapid.

### Cell Phenotypic Effects

To assess the time course associated with inhibition of growth by Compound A, an extended duration growth-death assay was performed in a subset of lymphoma cell lines.

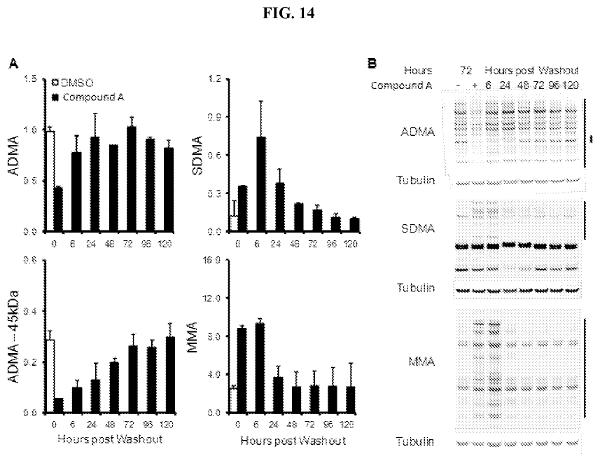

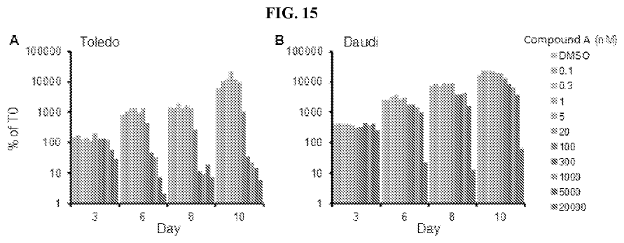

Similar to the 6-day proliferation assay described previously, the seeding density was optimized to ensure growth throughout the duration of the assay, and cell number was assessed by CTG at selected timepoints beginning from days 3-10. Growth inhibition was observed as early as 6 days and was maximal by 8 days in Toledo and Daudi lymphoma cell lines (FIG. 15).

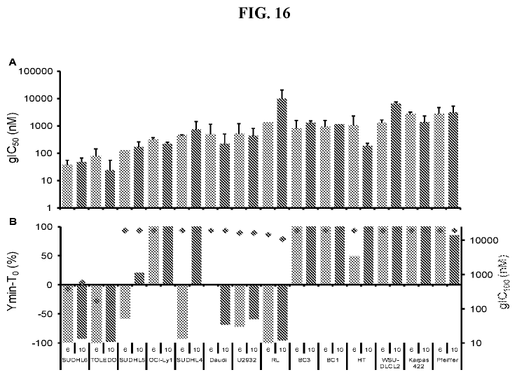

A larger set of cell lines was evaluated on days 6 and 10 to measure the effects of prolonged exposure to Compound A and determine whether cell lines that displayed a cytostatic response in the 6-day assay might undergo cytotoxicity at later timepoints. The extended time of exposure to Compound A had minimal effects on potency (gIC₅₀) or cytotoxicity (Yₘᵢₙ−T₀) across lymphoma cell lines evaluated (FIG. 16) indicating that 6-day proliferation evaluation could be utilized for assessment of sensitivity.

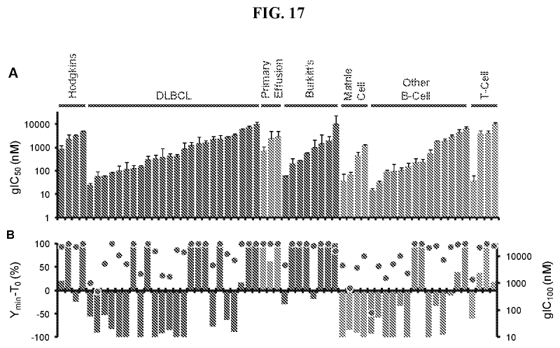

Given that growth inhibition was apparent at day 6 and prolonged exposure had minimal impact on potency or percent inhibition, a broad panel of lymphoma cell lines representing Hodgkin's and non-Hodgkin's subtypes was evaluated in the 6-day growth-death assay format (FIG. 17). All subtypes appeared equally sensitive in this format and many cell lines underwent cytotoxicity (as indicated by negative Yₘᵢₙ−T₀) independent of classification, suggesting that Compound A has anti-tumor effects in all subtypes of lymphoma evaluated.

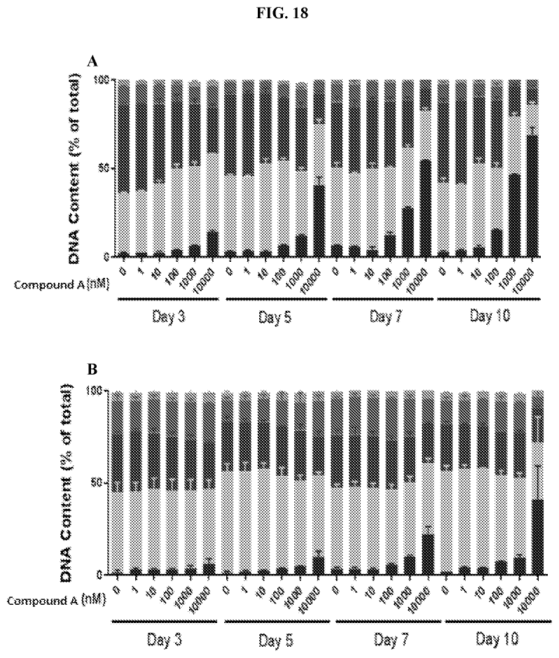

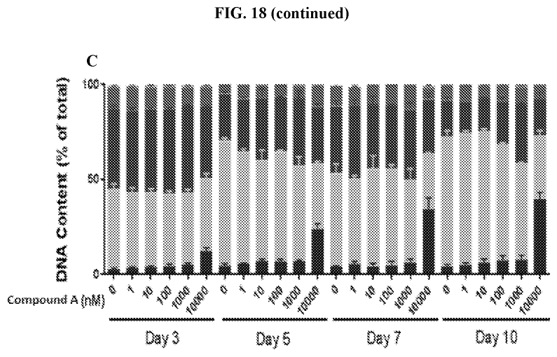

The proliferation assay results suggest that the inhibition of PRMT1 induces apparent cytotoxicity in a subset of lymphoma cell lines. To further elucidate this effect, the cell cycle distribution in lymphoma cell lines treated with Compound A was evaluated using propidium iodide staining followed by flow cytometry. Cell lines that showed a range of Yₘᵢₙ−T₀ and gIC₅₀ values in the 6-day proliferation assay were seeded at low density to allow logarithmic growth over the duration of the assay, and treated with varying concentrations of Compound A. Consistent with the growth-death assay results, an accumulation of cells in sub-G1 (<G1), indicative of cell death, was observed in Toledo cells in a time and dose dependent manner beginning after 3 days of treatment with Compound A concentrations ≥1000 nM (FIG. 18). By day 7, an increase in the sub-G1 population was apparent at concentrations ≥100 nM. In U2932 and OCI-Ly1, cell lines that underwent apparent cytostatic growth inhibition in the 6-day proliferation assay, this effect was only evident at 10 μM Compound A. No profound effect in any other cell cycle phase was revealed in this assay format.

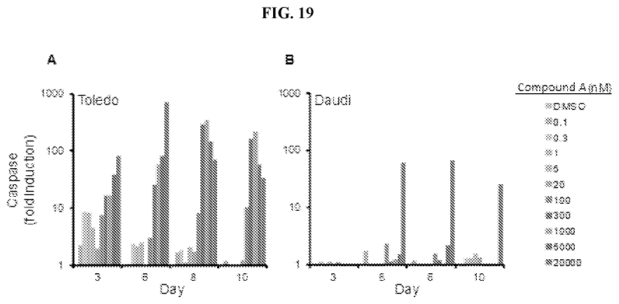

To confirm the FACS analysis of cell cycle, evaluation of caspase cleavage was performed as an additional measure of apoptosis during a 10-day timecourse. Seeding density was optimized to ensure consistent growth throughout the duration of the assay, and caspase activation was assessed using a luminescent Caspase-Glo 3/7 assay (Promega). Caspase-Glo 3/7 signal was normalized to cell number (assessed by CTG) and shown as fold-induction relative to control (DMSO treated) cells. Caspase 3/7 activity was monitored over a 10-day timecourse in DLBCL cell lines showing cytotoxic (Toledo) and cytostatic (Daudi) responses to Compound A (FIG. 19). Consistent with the profile observed in the growth-death assay, the Toledo cell line showed robust caspase activation concurrent with decreases in cell number at all timepoints, while induction of caspase activity in the Daudi cell line was less pronounced and limited to the highest concentrations of Compound A.

Together with the cell cycle profiles, these data indicate that Compound A induces caspase-mediated apoptosis in the Toledo DLBCL cell line, suggesting the cytotoxicity observed in other lymphoma cell lines may reflect activation of apoptotic pathways by Compound A. Gene expression patterns and somatic alterations were compared between cell lines that undergo cytotoxic and cytostatic responses upon Compound A treatment to identify predictive biomarkers associated with cytotoxicity. Although this analysis revealed no apparent correlation, examination of literature together with an approach to explore rational combinations identified deletion of the 5-Methylthioadenosine phosphorylase (MTAP) gene as a potential marker of cytotoxicity.

### Anti-Tumor Effects in Mouse Xenografts

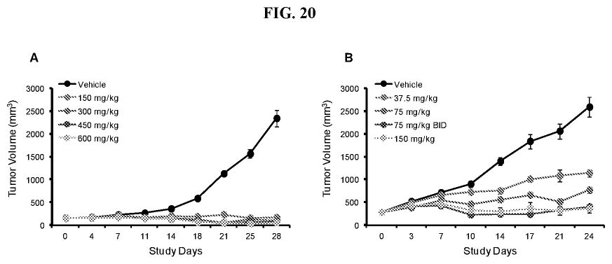

The effect of Compound A on tumor growth was assessed in a Toledo (human DLBCL) xenograft model. Female SCID mice bearing subcutaneous Toledo tumors were weighed, tumors were measured with callipers, and mice were block randomized according to tumor size into treatment groups of 10 mice each. Mice were dosed orally with either vehicle or Compound A (150 mg/kg-600 mg/kg) for 28 days daily. Throughout the study, mice were weighed and tumor measurements were taken twice weekly. Significant tumor growth inhibition (TGI) was observed at all doses and regressions were observed at doses ≥300 mg/kg (FIG. 20, Table 5). There was no significant body weight loss in any dose group.

Given that complete TGI was observed at all doses evaluated, a second study was performed to test the anti-tumor effect of Compound A at lower doses as well as to compare twice daily (BID) dosing relative to daily (QD). In this second study, mice were dosed orally with either vehicle or Compound A (37.5 mg/kg-150 mg/kg) for 24 days QD or 75 mg/kg BID. In this study, BID administration of 75 mg/kg resulted in the same TGI as 150 mg/kg (95% and 96%, respectively) while ≤75 mg/kg QD resulted in partial TGI (≤79%) (FIG. 20, Table 5). No significant body weight loss was observed in any dose group. These data suggest that either BID or QD dosing with the same total daily dose should result in similar efficacy.

### Additional Tumor Types

### AML

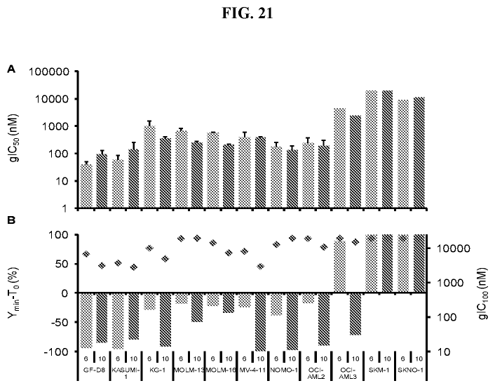

In addition to lymphoma cell lines, Compound A had potent, cytotoxic activity in a subset of AML cell lines examined in the 6-day proliferation assay (Table 3). Eight of 10 cell lines had gIC₅₀ values <2 μM, and Compound A induced cytotoxicity in 5 cell lines. Although PRMT1 interacts with the AML-ETO fusion characteristic of the M2 AML subtype (Shia, W. J. et al. PRMT1 interacts with AML-ETO to promote its transcriptional activation and progenitor cell proliferative potential. Blood 119, 4953-4962, doi:10.1182/blood-2011-04-347476 (2012)), cell lines carrying this fusion protein (Kasumi-1 and SKNO-1) were not the only lines showing sensitivity to Compound A as measured by gIC₅₀ or that underwent cytotoxicity (Table 3, FIG. 21), therefore, the presence of this oncogenic fusion protein does not exclusively predict sensitivity of AML cell lines to Compound A.

TABLE 3
Summary of Compound A activity in AML cell lines

| Cell Line | gIC₅₀ (μM) | gIC₁₀₀ (μM) | Ymin-T₀ | Subtype |
|---|---|---|---|---|
| HL-60 | 0.02 ± 0.01 | 6.38 ± 12.83 | −33.4 | M3 |
| MV-4-11 | 0.12 ± 0.08 | 14.55 ± 4.27 | 565.6 | M5 |
| MOLM-13 | 0.21 ± 0.01 | 8.64 ± 0.39 | −100.0 | M5 |
| SKM-1 | 0.22 ± 0.11 | 11.61 ± 5.52 | −19.1 | M5 |
| KASUMI- | 0.36 ± 0.25 | 18.88 ± 10.55 | −17.7 | M2 |
| MOLM-16 | 0.65 ± 0.01 | 9.69 ± 10.58 | −68.6 | M0 |
| OCI- | 0.87 ± 0.14 | 29.33 ± 0.00 | 523.2 | M4 |
| TF-1 | 1.67 ± 0.36 | 29.33 ± 0.00 | 788.1 | M6 |
| NOMO-1 | 3.85 ± 2.10 | 29.33 ± 0.00 | 259.1 | M5 |
| SHI-1 | 4.29 ± 3.52 | 29.33 ± 0.02 | 292.0 | M5 |

Similar to studies in lymphoma, a set of cell lines was evaluated on days 6 and 10 to measure the effects of prolonged exposure to Compound A and determine whether AML cell lines that displayed a cytostatic response in the 6-day assay might undergo cytotoxicity at later timepoints. Consistent with the lymphoma result, extending time of exposure to Compound A had minimal effects on potency (gIC₅₀) or cytotoxicity (Yₘᵢₙ−T₀) across AML cell lines evaluated (FIG. 21).

### Renal Cell Carcinoma

Renal cell carcinoma cell lines had among the lowest median gIC₅₀ compared with other solid tumor types. Although none of the lines tested showed a cytotoxic response upon treatment with Compound A, all showed complete growth inhibition and 6 of 10 had gIC₅₀ values ≤2 μM (Table 4). 7 of the 10 lines profiled represent clear cell renal carcinoma (ccRCC), the major clinical subtype of renal cancer.

TABLE 4
Summary of Compound A anti-proliferative effects in renal
cell carcinoma cells

| Cell Line | gIC₅₀ (μM) | Ymin-T₀ | Subtype |
|---|---|---|---|
| ACHN | 0.10 ± 0.05 | 96.5 | ccRCC |
| CAKI-1 | 0.28 ± 0.23 | 178.7 | ccRCC |
| G-401 | 0.35 ± 0.04 | 353.7 | Wilm's |
| 786-O | 0.59 ± 0.41 | 643.7 | ccRCC |
| SK-NEP-1 | 1.43 ± 0.86 | 25.3 | Wilm's |
| 769-P | 1.89 ± 0.82 | 119.0 | ccRCC |
| A498 | 2.73 ± 2.81 | 313.4 | ccRCC |
| G-402 | 2.89 ± 2.05 | 92.6 | Leiomyoblastoma |
| SW156 | 3.51 ± 2.01 | 346.7 | ccRCC |
| CAKI-2 | 4.23 ± 1.51 | 169.6 | ccRCC |

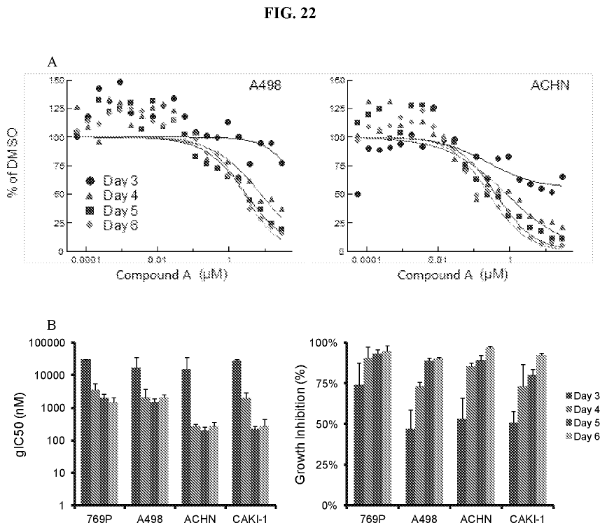

To assess the time course of growth inhibition in renal carcinoma cell lines by Compound A, cell growth was assessed by CTG in a panel of 4 ccRCC cell lines at days 3,4,5, and 6 (FIG. 22). The largest shift in activity occurred between days 3 and 4, where all cell lines showed decreases gIC₅₀ values and increases growth inhibition. Potency of Compound A (assessed by gIC₅₀) was maximal by 4 days in 3 of 4 lines and did further not change through the 6 day assay duration. Additionally, percent growth inhibition reached 100% in all cell lines evaluated. Therefore, maximal growth inhibition in ccRCC cell lines was apparent within the 6-day growth window utilized in the cell line screening strategy.

Caspase activation was evaluated during the proliferation timecourse and, consistent with the lack of overt cytotoxicity as indicated by the Yₘᵢₙ−T₀ values, caspase cleavage only occurred at the highest concentration (30 μM) indicating that apopotosis may have a minimal contribution to the overall growth inhibitory effect induced by Compound A in ccRCC cell lines.

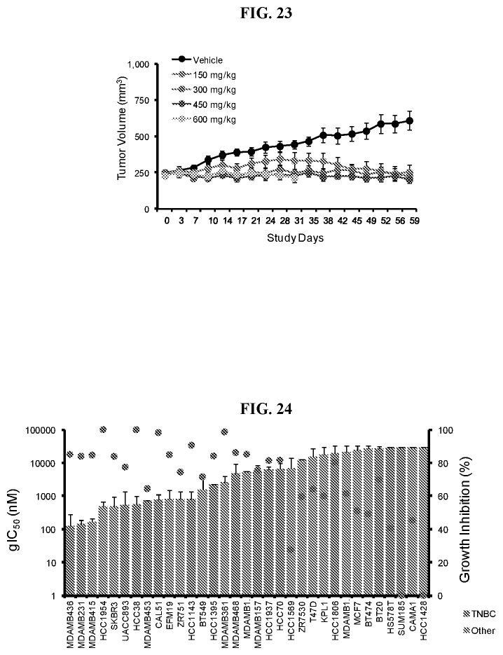

The effect of Compound A on tumor growth was assessed in mice bearing human renal cell carcinoma xenografts (ACHN). Female SCID mice bearing subcutaneous ACHN cell line tumors were weighed and tumors were measured by callipers and block randomized according to tumor size into treatment groups of 10 mice each. Mice were dosed orally with either vehicle or Compound A (150 mg/kg-600 mg/kg) for up to 59 days daily. Throughout the study, mice were weighed and tumor measurements were taken twice weekly. Significant tumor growth inhibition was observed at all doses and regressions were observed at doses ≥300 mg/kg. Significant body weight loss was observed in animals treated with 600 mg/kg daily and, therefore, that dosing group was terminated on day 31 (FIG. 23, Table 5).

TABLE 5
Efficacy of Compound A in vivo

| Cell Line | | | | | Body weight |
|---|---|---|---|---|---|
| (Tumor | Dose | | TGI | | Difference |
| Type) | (mg/kg) | | (Regression) | Day | (vs. vehicle) |
| Toledo | 150 | QD | 99%* | 28 | −4% |
| (DLBCL) | 300 | QD | 100%* (37%) | | −3% |
| | 450 | QD | 100%* (58%) | | −8% |
| | 600 | QD | 100%* (62%) | | −7% |
| Toledo | 37.5 | QD | 63%* | 25 | −5% |
| (DLBCL) | 75 | QD | 79%* | | −5% |
| | 75 | BID | 95%* | | −4% |
| | 150 | QD | 96%* | | −7% |
| ACHN | 150 | QD | 98%* | 59 | −3% |
| (ccRCC) | 300 | QD | 100%* (2%) | | −4% |
| | 450 | QD | 100%* (15%) | | −7% |
| | 600 | QD** | 100%* (6%) | | −17% |

*p < 0.05, two-tailed t-test
**600 QD arm of ACHN efficacy study was terminated at day 31

Together, these data suggest that 100% TGI can be achieved at similar doses in subcutaneous xenografts of human solid and hematologic tumors.

### Breast Cancer

Breast cancer cell lines displayed a range of sensitivities to Compound A and in many cases, showed partial growth inhibition in the 6-day proliferation assay (FIG. 24). Cell lines representing triple negative breast cancer (TNBC) had slightly lower median gIC₅₀ values compared with non-TNBC cell lines (3.6 μM and 6.8 μM for TNBC and non-TNBC, respectively).

Since the effect on proliferation by Compound A was cytostatic and did not result in complete growth inhibition in the majority of breast cancer cell lines, an extended duration growth-death assay was performed to determine whether the sensitivity to Compound A would increase with prolonged exposure. In 7/17 cell lines tested there was an increase in percent maximal inhibition by ≥10% and a ≥2-fold decrease in gIC₅₀ (FIG. 25). In the prolonged exposure assay, 11/17 cell lines had gIC₅₀≤2 μM (65%) while 7/17 (41%) met this criteria in the 7 day assay format.

### Melanoma

Among solid tumor types, Compound A had the most potent anti-proliferative effect in melanoma cell lines (FIG. 11). Six of 7 lines assessed had gIC₅₀ values less than 2 μM (Table 6). The effect of Compound A was cytostatic in all melanoma lines, regardless of gIC₅₀ value.

TABLE 6
Summary of Compound A Activity in Melanoma Cell Lines

| | | | Yₘᵢₙ- |
|---|---|---|---|
| Cell Line | gIC₅₀ (μM) | gIC₁₀₀ (μM) | T₀ |
| A375 | 0.05 ± 0.03 | 29.33 ± 0.00 | 91.9 |
| SK-MEL-5 | 0.09 ± 0.03 | 27.09 ± 3.92 | 31.8 |
| IGR-1 | 0.27 ± 0.14 | 29.33 ± 0.00 | 507.0 |
| SK-MEL-2 | 0.28 ± 0.14 | 22.37 ± | 35.9 |
| COLO741 | 0.43 ± 0.37 | 28.55 ± 1.40 | 12.5 |
| HT144 | 3.46 ± 2.68 | 29.33 ± 0.00 | 124.9 |
| MDA-MB-435S | 29.36 ± | 29.33 ± 0.00 | 19.1 |

### Example 2

### Predictive Biomarkers

The rank order of sensitivity of cell lines to Compound A by gIC50 and association with somatic alterations or gene expression was examined using genomic data available through Cancer Cell Line Encylopedia (CCLE). In addition, lymphoma lines were stratified by their ability to undergo a cytotoxic response to Compound A. No apparent correlation to any cancer relevant alteration could be determined using this approach, potentially due to the broad activity of Compound A in cell culture. Therefore, a rational approach was investigated based on the combination activity observed with PRMT5 inhibition.

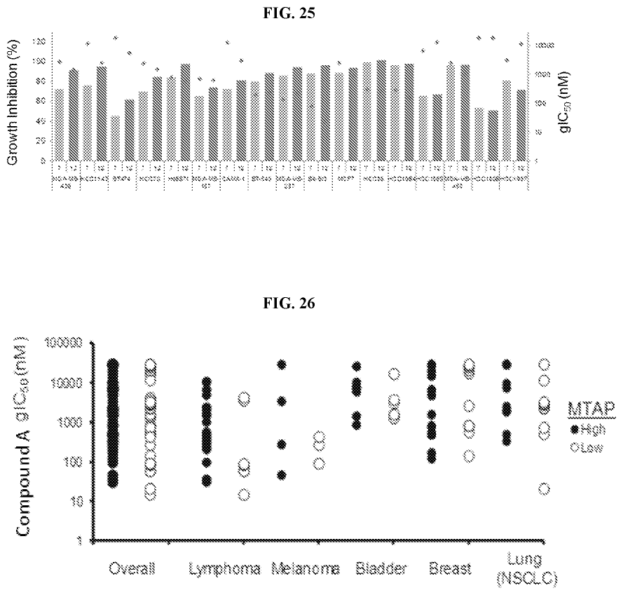

Recent studies described a mechanism by which loss of the 5-Methylthioadenosine phosphorylase (MTAP) gene may inhibit endogenous PRMT5 in tumor cells. The MTAP gene is frequently deleted in cancers including 40% of glioblastoma, 25% of melanoma and pancreatic adenocarcinoma, and 15% of non-small cell lung carcinoma. (Mavrakis, K. J. et al., Disordered methionine metabolism in MTAP/CDKN2A-deleted cancers leads to dependence on PRMT5. Science 351, 1208-1213, doi:10.1126/science.aad5944 (2016); Marjon, K. et al., MTAP Deletions in Cancer Create Vulnerability to Targeting of the MAT2A/PRMT5/RIOK1 Axis. Cell Rep 15, 574-587, doi:10.1016/j.celrep.2016.03.043 (2016); Kryukov, G. V. et al., MTAP deletion confers enhanced dependency on the PRMT5 arginine methyltransferase in cancer cells. Science 351, 1214-1218, doi:10.1126/science.aad5214 (2016)). Loss of MTAP leads to increased levels of the metabolite, methylthioadenosine (MTA), shown to inhibit PRMT5 biochemical activity, resulting in lower cellular levels of SDMA (Mavrakis, K. J. et al., Disordered methionine metabolism in MTAP/CDKN2A-deleted cancers leads to dependence on PRMT5. Science 351, 1208-1213, doi:10.1126/science.aad5944 (2016); Marjon, K. et al., MTAP Deletions in Cancer Create Vulnerability to Targeting of the MAT2A/PRMT5/RIOK1 Axis. Cell Rep 15, 574-587, doi:10.1016/j.celrep.2016.03.043 (2016); Kryukov, G. V. et al., MTAP deletion confers enhanced dependency on the PRMT5 arginine methyltransferase in cancer cells. Science 351, 1214-1218, doi:10.1126/science.aad5214 (2016)). Given the combined effects of Compound A and a PRMT5 inhibitor on growth inhibition of cancer cell lines, MTAP deletion may offer a scenario in which endogenous PRMT5 is partially inhibited, thereby sensitizing cells to PRMT1 inhibition and lowering the concentration of Compound A required for efficacy. In a tumor type agnostic manner, MTAP loss did not correlate with Compound A sensitivity. However, lower median gIC50 associated with Compound A treatment correlated with MTAP deletion (>5-fold difference relative to MTAP proficient cell lines) in lymphoma and melanoma cell lines (FIG. 26). While these differences were not statistically significant due, in part, to low numbers (N) within select tumor types, these observations contributed to the development of a predictive biomarker hypothesis.

Moreover, in lymphoma, cell lines with MTAP deletion undergo cytotoxicity in response to Compound A as indicated by a shift from a positive to negative Ymin−T0 (Table 7).

TABLE 7
Median growth parameters of cancer cell
lines, by tumor type and MTAP status

| | gIC₅₀, μM | | gIC₁₀₀, μM | | % Yₘᵢₙ − T₀ | |
|---|---|---|---|---|---|---|
| MTAP | High | Low | High | Low | High | Low |
| Lymphoma | 0.6 | 0.1 | 24.4 | 5.3 | 17 | −89 |
| Melanoma | 1.9 | 0.3 | 29.4 | 29.0 | 214 | 53 |
| Bladder | 7.6 | 1.6 | 29.3 | 29.3 | 658 | 257 |
| Lung | 5.0 | 3.0 | 29.3 | 29.3 | 141 | 237 |
| (NSCLC) | | | | | | |
| Breast | 4.9 | 10.2 | 29.4 | 29.3 | 174 | 188 |

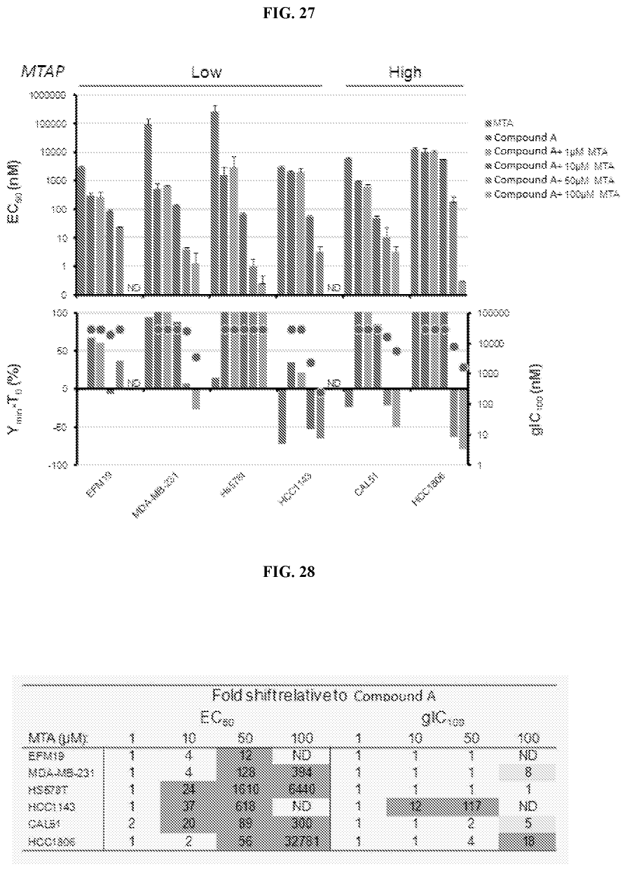

Recent publications highlighting a mechanism by which MTA can inhibit PRMT5 also evaluated levels of MTA in cultured cells. While there was some variation in MTAP proficient and deficient lines, overall MTA levels appeared to increase with time in culture (Kamatani, N. & Carson, D. A. Abnormal regulation of methylthioadenosine and polyamine metabolism in methylthioadenosine phosphorylase-deficient human leukemic cell lines. Cancer Res 40, 4178-4182 (1980)). This leads to the hypothesis that a 6-day proliferation assay used to investigate the relationship between MTAP expression and sensitivity to Compound A may not sufficiently reveal a correlation if MTA levels do not reach a level required to inhibit PRMT5 during the course of the assay. To further investigate the potential of elevated MTA levels to combine with Compound A to inhibit cancer cell growth, fixed concentrations of exogenous MTA (1, 10, 50, or 100 μM) were tested with a 20-point titration of Compound A in a 6-day proliferation assay. Six breast cancer cell lines were chosen that did not show increased sensitivity to Compound A through MTAP deficiency. Due to the effect of the highest concentrations of MTA on the growth window, the EC50 values were used to compare potency rather than gIC₅₀. A decrease in EC₅₀ of Compound A (>10-fold) was apparent in every cell line evaluated with at least one concentration of MTA (FIG. 27). Additionally, a shift from cytostatic to cytotoxic (negative Ymin−T0) was apparent in 3 of 5 cell lines that had cytostatic or no response to either single agent (FIG. 28).

Together, this data suggest that tumor specific loss of MTAP can reveal increased sensitivity to Compound A through increase in an endogenous inhibitor of PRMT5. Since elevated MTA levels in MTAP deleted tumors would inhibit PRMT5, MTAP deletion may have potential utility as a predictive biomarker of Compound A sensitivity. To determine whether MTA levels reach sufficient concentrations to inhibit PRMT5 in MTAP null tumors, evaluation of MTA levels in cell lines with MTAP deletion as well as in primary tumors is currently underway.

1. A method of treating cancer in a human in need thereof, the method comprising determining
a. the level of 5-Methytthioadenosine phosphorylase (MTAP) polynucleotide or polypeptide or
b. the presence or absence of a mutation in MTAP in a sample from the human, and administering to the human an effective amount of a Type I protein arginine methyltransferase (Type I PRMT) inhibitor if the level of the MTAP polynucleotide or polypeptide is decreased relative to a control or if a mutation in MTAP polynucleotide or polypeptide is present, thereby treating the cancer in the human.

2. A method of inhibiting proliferation of a cancer cell in a human in need thereof, the method comprising administering to the human an effective amount of a Type I protein arginine methyltransferase (Type I PRMT) inhibitor, thereby inhibiting proliferation of the cancer cell in the human, wherein the cancer cell has a mutation in 5-Methytthioadenosine phosphorylase (MTAP) and/or a decreased level of a MTAP polynucleotide or polypeptide relative to a control.

3. (canceled)

4. (canceled)

5. The method of claim 1, wherein the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor.

6. The method of claim 1, wherein the Type I PRMT inhibitor is a compound of Formula (I): 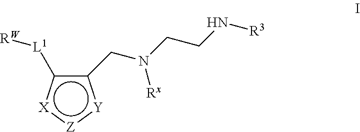
or a pharmaceutically acceptable salt thereof,
wherein
X is N, Z is NR⁴, and Y is CR⁵; or
X is NR⁴, Z is N, and Y is CR⁵; or
X is CR⁵, Z is NR⁴, and Y is N; or
X is CR⁵, Z is N, and Y is NR⁴;
R^{X} is optionally substituted C₁₋₄ alkyl or optionally substituted C₃₋₄ cycloalkyl;
L₁ is a bond, —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, —SO₂N(R^{B})—, or an optionally substituted C₁₋₆ saturated or unsaturated hydrocarbon chain, wherein one or more methylene units of the hydrocarbon chain is optionally and independently replaced with —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, or —SO₂N(R^{B})—;
each R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom;
each R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or an R^{B} and R^{W} on the same nitrogen atom may be taken together with the intervening nitrogen to form an optionally substituted heterocyclic ring;
R^{W} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; provided that when L₁ is a bond, R^{W} is not hydrogen, optionally substituted aryl, or optionally substituted heteroaryl;
R³ is hydrogen, C₁₋₄ alkyl, or C₃₋₄ cycoalkyl;
R⁴ is hydrogen, optionally substituted C₁₋₆ alkyl, optionally substituted C₂₋₆ alkenyl, optionally substituted C₂₋₆ alkynyl, optionally substituted C₃₋₇ cycloalkyl, optionally substituted 4- to 7-membered heterocyclyl; or optionally substituted C₁₋₄ alkyl-Cy;
Cy is optionally substituted C₃₋₇ cycoalkyl, optionally substituted 4- to 7-membered heterocyclyl, optionally substituted aryl, or optionally substituted heteroaryl; and
R⁵ is hydrogen, halo, —CN, optionally substituted C₁₋₄ alkyl, or optionally substituted C₃₋₄ cycoalkyl.

7. The method of claim 6, wherein the Type I PRMT inhibitor is a compound of Formula (II): 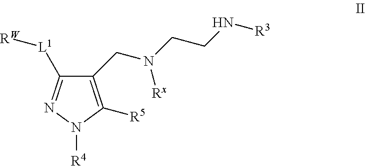
or a pharmaceutically acceptable salt thereof.

8. The method of claim 6, wherein the Type I PRMT inhibitor is a compound of Formula (I) or (II) wherein -L₁-R^{W} is optionally substituted carbocyclyl.

9. The method of claim 1, wherein the Type I PRMT inhibitor is Compound A: 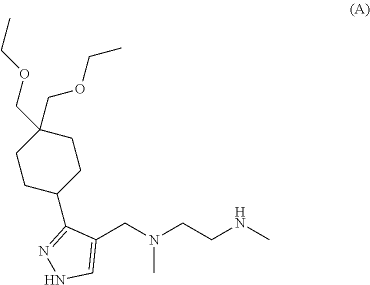 [SMILES: CCOCC1(COCC)CCC(c2n[nH]cc2CN(C)CCNC)CC1]
or a pharmaceutically acceptable salt thereof.

10. The method of claim 1, wherein the mutation is an MTAP deletion.

11. The method of claim 1, wherein the sample comprises a cancer cell.

12. The method of claim 1, wherein the cancer is a solid tumor or hematological cancer.

13. The method of claim 2, wherein the cancer cell is a solid tumor cancer cell or hematological cancer cell.

14. The method of claim 1, wherein the cancer is lymphoma, acute myeloid leukemia (AML), kidney, melanoma, breast, bladder, colon, lung, or prostate.

15. The method of claim 2, wherein the cancer cell is a lymphoma cell, acute myeloid leukemia (AML) cell, kidney cancer cell, melanoma cell, breast cancer cell, bladder cancer cell, colon cancer cell, lung cancer cell, or prostate cancer cell.

16. The method of claim 2, wherein the decreased level of MTAP polynucleotide or polypeptide or the mutation in MTAP increases the level of methythioadenosine (MTA) in the cancer cell such that the activity of protein arginine methyltransferase 5 (PRMT5) is inhibited.

17. The method of claim 2, wherein the decreased level of MTAP polynucleotide or polypeptide or the mutation in MTAP in the cancer cell increases sensitivity of the cancer cell to the Type 1 PRMT inhibitor.

18. The method of claim 1, wherein both a and b are determined.

19. The method of claim 1, further comprising administering one or more additional anti-neoplastic agents.

20. (canceled)

21. (canceled)

22. (canceled)

23. (canceled)


SEQUENCE LISTING (2 sequences)

SEQ ID NO: 1 - protein, 283 aa, Homo sapiens
MASGTTTTAVKIGIIGGTGLDDPEILEGRTEKYVDTPFGKPSDALILGKIKNVDCVLLARHGRQHTIMPSKVNYQANIWALKEEGCTHVIVTTACGSLREEIQPGDIVIIDQFIDRTTMRPQSFYDGSHSCARGVCHIPMAEPFCPKTREVLIETAKKLGLRCHSKGTMVTIEGPRFSSRAESFMFRTWGADVINMTTVPEVVLAKEAGICYASIAMATDYDCWKEHEEAVSVDRVLKTLKENANKAKSLLLTTIPQIGSTEWSETLHNLKNMAQFSVLLPRH

SEQ ID NO: 2 - DNA, 4937 bp, Homo sapiens
ctccgcactgctcactcccgcgcagtgaggttggcacagccaccgctctgtggctcgcttggttcccttagtcccgagcgctcgcccactgcagattcctttcccgtgcagacatggcctctggcaccaccaccaccgccgtgaagattggaataattggtggaacaggcctggatgatccagaaattttagaaggaagaactgaaaaatatgtggatactccatttggcaagccatctgatgccttaattttggggaagataaaaaatgttgattgcgtcctccttgcaaggcatggaaggcagcacaccatcatgccttcaaaggtcaactaccaggcgaacatctgggctttgaaggaagagggctgtacacatgtcatagtgaccacagcttgtggctccttgagggaggagattcagcccggcgatattgtcattattgatcagttcattgacaggaccactatgagacctcagtccttctatgatggaagtcattcttgtgccagaggagtgtgccatattccaatggctgagccgttttgccccaaaacgagagaggttcttatagagactgctaagaagctaggactccggtgccactcaaaggggacaatggtcacaatcgagggacctcgttttagctcccgggcagaaagcttcatgttccgcacctggggggcggatgttatcaacatgaccacagttccagaggtggttcttgctaaggaggctggaatttgttacgcaagtatcgccatggcgacagattatgactgctggaaggagcacgaggaagcagtttcggtggaccgggtcttaaagaccctgaaagaaaacgctaataaagccaaaagcttactgctcactaccatacctcagatagggtccacagaatggtcagaaaccctccataacctgaagaatatggcccagttttctgttttattaccaagacattaaagtagcatggctgcccaggagaaaagaagacattctaattccagtcattttgggaattcctgcttaacttgaaaaaaatatgggaaagacatgcagctttcatgcccttgcctatcaaagagtatgttgtaagaaagacaagacattgtgtgtattagagactcctgaatgatttagacaacttcaaaatacagaagaaaagcaaatgactagtaaacatgtgggaaaaaatattacattttaagggggaaaaaaaaacccaccattctcttctccccctattaaatttgcaacaataaagggtggagggtaatctctactttcctatactgccaaagaatgtgaggaagaaatgggactctttggttatttattgatgcgactgtaaattggtacagtatttctggagggcaatttggtaaaatgcatcaaaagacttaaaaatacggacgtactttgtgctgggaactctacatctagcaatttctctttaaaaccatatcagagatgcatacaaagaattatatataaagaagggtgtttaataatgatagttataataataaataattgaaacaatctgaatcccttgcaattggaggtaaattatgtcttagttataattagattgtgaatcagccaactgaaaatcctttttgcatatttcaatgtcctaaaaagacacggttgctctatatatgaagtgaaaaaaggatatggtagcattttatagtactagttttgctttaaaatgctatgtaaatatacaaaaaaactagaaagaaatatatataaccttgttattgtatttgggggagggatactgggataatttttattttctttgaatctttctgtgtcttcacatttttctacagtgaatttaatcaaatagtaaagttgttgtaaaaataaaagtggatttagaaagatccagttcttgaaaacactgtttctggtaatgaagcagaatttaagttggtaatattaaggtgaatgtcatttaagggagttacatctttattctgctaaagaagaggatcattgatttctgtacagtcagaacagtacttgggtttgcaacagctttctgagaaaagctaggtgtttaatagtttaactgaaagtttaactatttaaaagactaaatgcacattttatggtatctgatattttaaaaagtaatgtttgattctcctttttatgagttaaattattttatacgagttggtaatttttgctttttaataaagtggaagcttgcttttttaactctttttttattgttattttatagaaatgctttttgttggccgggcacagttgctcatccatgtaatcccagcactgtgggaggccgagacgggtggatcacaaggtcaggagatcgagaccatcctggctaatgcgttgaaactccgtctctactaaaaatacaaaaaattagctgggcgtggtggtgggcacctgtagtcccagctactcaggaggctgaggcaggagaatggtgtgaacctgggaggtggagcttgcagtgagcagagcttgcagtgagacgagcttgtgccactgcactccagcctgggcaacagagtaagactcagtctcaaaaaaaaaaaaaagagtgaaatgctttttgtttgcttcagttttttatcatggggagatctttttcctcagaattgttttcttttcactgtaggctattacaggatacttcaggatcaagatacagaaccttttatttaaagagtttgtaaagtcaatgtgtttgtttgtgtctctgagattgacttcaagataataagctgctaattgtaaacaaaacagttaccctccagtattaatatgactcattagtgtgagccatttgggtcaagtatgattatgacccttggacttcctgatgtagtattaaatttcaactctggttatccattagcaatctgtagagaacttaatgaacctgaacccaggcttctctagctctggtaacgtgtgattgttttcactacaatatgatacatagatggtaccttacttttcctcattcttaataggtgtctaagaatgtcagggcaaaagtatgggcatttttcttgctatgttcagaaagtacagttctctccaacttgcagaggtacttttcttgattaaatagccttctctagcaacatcattttcagactaactaaatgaatgcagtatactcttttctttgttctcaatcattcactccttatgcaaagccaatataattttcctcataccttatgcttgaggatattgttgaagaacacttcctggaacacttctcacttgtgatgctgtactaattttttttttttaatttaagctagtatactaagtgaacaccatggtcagttgtgagcattttggtttccgcaaaggatggatggtgagcatcatgggaaagctgtagtttagtgacttagcccttagtgattaatagatttgcatgtacatagaagtctttgttggccttataatctgctgttatatttggcatggattttcatggttttgagaatgacatcctggccctgtggtccccgagggtcatggtccttgtgacctggcccctgttcactgcccccttcgctagcacgagttgctgtgcagggctggaggtagctaccatggcttgtttcaaggaaggaaactctggtacggtggcaccctcaggagtggaggacagtgaacttccttgaagagggagtgactaaggtgacctccaacctgccctgagccagctgccctgcaggtgccacgtgagcctgctctggcatccacaggatgctcctggagcctcttctctggctgctacctcagggcatggttgtggccccaccaacacctattttccaaataattattcattcttgtgacagtggcctgaacatgtttttaattttctcaacaagcatttagccagcacttatccagtgaaacaatttgataaggtttcaaggagtatctgatgggttaggaagtcacgaaatgaggagttcttgccacatttgcagagtccctccttgataaggtttggcggtgtccccacccaaatctcatgttgaattgtagttcccataatccccacatgttgtgggagggacccagtgggaggtaattaaatcatgggggtggttacccccacactgctgttctcatgatactgagttctcacaagtcctgtttgttttataaggggcttttcccccttttgctcaacacttcttcctgccatcatgtgaagaaggacgtgtttgtttccccttctgccacgattgtaagtttcctgaggccttcccagctatgtggaactgtgagttaattaaacctctttcctttataaattacccagtcatgggcagtcctttacagcagcatgagaatggactaatacactcctcaaatgttttgaagattgttgcaccttggaactaccagtgtgcacacaatctggctcaatgtatatattggcccagcaaggcaaagaactgaagttccaggatggaagaacctgtgttctcctcataatagtatagaataattcaagataggcaagaaggacagcagtaaatgaagaccatggaagaaaagaaggaatgccaaagatcgaggaaatctaccaagactagtagggtagtccagaagaagctgtttcagggcctgttgccagctatgcctttgagaacctcgggatcccaaagaatgaggggaatttcttcagaaagacaatctcggcatgcattatttctttgttttgaagattcactcatgttgcatgcatctgtagcttgtgccttttttattgcctagtagtattctgtcatatgcctatcttacaatttgattatctattcacctgttgatgaatgtttgaattttttccatttgaggaattttatgaataaagctgctataagcatgaaaaaaaaaaaaaaaaaa